(19) United States
(12) Patent Application Publication
(10) Pub. No.: US 2025/0085263 A1
(43) Pub. Date: Mar. 13, 2025

(54) METHOD TO ESTABLISH A DETECTABLE LEAK SOURCE LOCATION

(71) Applicant: Schlumberger Technology Corporation, Sugar Land, TX (US)

(72) Inventors: Kashif Rashid, Cambridge, MA (US); Lukasz Zielinski, Cambridge, MA (US); Andrew J. Speck, Cambridge, MA (US)

(21) Appl. No.: 18/754,234

(22) Filed: Jun. 26, 2024

(30) Foreign Application Priority Data
Sep. 8, 2023 (WO) PCT/US2023/032307

(51) Int. Cl. G01N 33/00; G01M 3/04; G01P 5/00

(52) U.S. Cl. CPC G01N 33/0075; G01M 3/04; G01P 5/001

(57) ABSTRACT

Embodiments presented provide for a method for detecting emissions. The method establishes a map that is used with prevailing wind conditions to establish a point source location for methane gas emissions.

### CROSS-REFERENCE TO RELATED APPLICATIONS

The present application is a continuation-in-part application of International Patent Application Number PCT/US2023/032307 filed on Sep. 8, 2023, entitled “A Method to Establish A Detectable Leak Source Location”, which claims priority to U.S. Provisional Patent Application 63/375,115, filed Sep. 9, 2022, the entirety of which is incorporated by reference.

### FIELD OF THE DISCLOSURE

Aspects of the disclosure relate to environmental monitoring. More specifically, aspects of the disclosure relate to a method to establish a detectable methane leak source location (or coverage) map given the prevailing wind conditions encountered, and the known locations and characteristics of the deployed methane sensors.

### BACKGROUND

Processing of gases is an integral part of modern society. Some gases are harmless to the environment, while other gases can cause significant changes and complications to ecosystems. During the transport and handling of gases, leaks may occur from time to time. These leaks may have serious consequences according to the type and size of the leak.

One of the more prevalent gases that is transported for processing is methane. Methane has several properties that lend itself to use in industrial settings and can be prized in certain applications. Methane is combustible; therefore, leaks can be problematic. Methane is also a greenhouse gas; contributing to unwanted fugitive emissions when it escapes.

Conventional leak identification merely involves workers using a gas analyzer and walking a path that the pipeline travels to see if any leaks have developed. Such leak analyzing techniques are expensive over time as repeated trips must be accomplished. There is a need to effectively monitor methane leaks or emissions in an environment without the constant, operator, time, expenditure of conventional techniques.

There is a need to provide an apparatus and methods that are easier to operate and less time consuming than conventional apparatus and methods.

There is a further need to provide apparatus and methods that do not have the drawbacks discussed above.

There is a still further need to reduce economic costs associated with operations and apparatus described above with conventional tools and methods.

### SUMMARY

So that the manner in which the above recited features of the present disclosure can be understood in detail, a more particular description of the disclosure, briefly summarized below, may be had by reference to embodiments, some of which are illustrated in the drawings. It is to be noted that the drawings illustrate only typical embodiments of this disclosure and are therefore not to be considered limiting of its scope, for the disclosure may admit to other equally effective embodiments without specific recitation. Accordingly, the following summary provides just a few aspects of the description and should not be used to limit the described embodiments to a single concept.

In one example embodiment, a method is disclosed. The method may comprise obtaining wind prevailing conditions for a site and obtaining sensor data information for the site. The method may also be performed to further comprise processing the wind prevailing conditions and sensor data information to produce an event detection and record generation event and storing the event detection and record event. The method may also comprise obtaining a facility map and processing the facility map, the stored event detection and record event to produce a potential source of emissions.

In another example embodiment, a method for identifying a source of emissions for a given site is disclosed. The method may comprise obtaining wind prevailing conditions for the site. The method may also comprise obtaining sensor data information for the site. The method may also comprise processing the wind prevailing conditions and sensor data information to produce a detection and record generation event. The method may also comprise storing the detection and record event and obtaining a facility map. The method may also comprise processing the facility map, the stored event detection, and record event, to produce a potential source of emissions for the site.

In another example embodiment, a method for developing a spatial coverage map for a site is disclosed. The method may comprise obtaining wind prevailing conditions for a site. The method may further comprise obtaining sensor data information for the site. The method may further comprise establishing an objective function measure for the site. The method may further comprise processing the wind prevailing conditions and sensor data information to produce a wind realization for the site. The method may further comprise storing the wind realization for the site. The method may further comprise establishing a coverage measure evaluation for the site based on the wind realization. The method may further comprise determining when the objective measure function has been achieved for the coverage measure evaluation. The method may further comprise ending the method when the objective function measure is successfully achieved. The method may further comprise establishing the coverage measure for a given wind realization and subsequently, establishing the mean coverage measure over all wind realizations. The method may further comprise continuing an optimization of the mean coverage measure evaluation with the given set of wind realizations until the optimal objective function measure is achieved.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

So that the manner in which the above recited features of the present disclosure can be understood in detail, a more particular description of the disclosure, briefly summarized above, may be had by reference to embodiments, some of which are illustrated in the drawings. It is to be noted; however, that the appended drawings illustrate only typical embodiments of this disclosure and are therefore not be considered limiting of its scope, for the disclosure may admit to other equally effective embodiments.

FIG. 1 is a high-level schema for source locating map identification.

FIG. 2 is a leak source detection map as a function of time.

FIG. 3 is a leak source detection schema.

FIG. 4 is a synthetic wind model and wind direction (top) and wind speed (bottom).

FIG. 5 is a graph of wind conditions, concentration readings and solar radiation of sensor 1, in one example embodiment of the disclosure.

FIG. 6 is a graph of wind conditions, concentration readings and solar radiation of sensor 2, in one example embodiment of the disclosure.

FIG. 7 is a graph of wind conditions, concentration readings and solar radiation of sensor 3, in one example embodiment of the disclosure.

FIG. 8 is a graph of wind conditions, concentration readings and solar radiation of sensor 4, in one example embodiment of the disclosure.

FIG. 9 is a graph of an inverse solution, designated by a star, for 24 hours, with a known source.

FIG. 10 is a graph of an objective evaluation of source (left hand side) and solution (right hand side).

FIG. 11 is a cone generation plot for sensor 1.

FIG. 12 is a cone generation plot for sensor 2.

FIG. 13 is a cone generation plot for sensor 4.

FIG. 14 is an inverse solution indicated by a star with a known source designated by a square.

FIG. 15 is an objective evaluation at the source (left hand side of figure) and solution (right hand side of figure).

FIG. 16 is an inversion solution (star) with a known source (square) for location 1.

FIG. 17 is an objective evaluation for location 1 of an objective evaluation at the source (left hand side) and solution (right hand side).

FIG. 18 is a graph for location 2 of an objective evaluation at the source (left hand side) and solution (right hand side).

FIG. 19 is an objective evaluation for location 2 at a source (left hand side) and solution (right hand side).

FIG. 20 is an inversion solution (star) with known source (square) for location 3.

FIG. 21 is an objective evaluation for location 3 of a source (left hand side) and solution (right hand side).

FIG. 22 is an inversion solution (star) with known source (square) for location 4.

FIG. 23 is an objective evaluation for location 4 of a source (left hand side) and solution (right hand side).

FIG. 24 is an inversion solution (star) with known source (square) for location 5.

FIG. 25 is an objective evaluation for location 5 of a source (left hand side) and solution (right hand side).

FIG. 26 is an inversion solution (star) with known source (square) for location 6.

FIG. 27 is an objective evaluation for location 6 of a source (left hand side) and solution (right hand side).

FIG. 28 is an inversion solution (star) with known source (square) for location 7.

FIG. 29 is an objective evaluation for location 7 of a source (left hand side) and solution (right hand side).

FIG. 30 is an inversion solution (star) with known source (square) for location 8.

FIG. 31 is an objective evaluation for location 8 of a source (left hand side) and solution (right hand side).

FIG. 32 is an inversion solution (star) with known source (square) for location 9.

FIG. 33 is an objective evaluation for location 9 of a source (left hand side) and solution (right hand side).

FIG. 34 is a graph of a spatial distance measure for six hours.

FIG. 35 is a graph of a spatial distance measure for twelve hours.

FIG. 36 is a graph of a spatial distance measure for 18 hours.

FIG. 37 is a graph of a spatial distance measure for 24 hours.

FIG. 38 is a graph of a rate distance measurement, in plan view, for a time interval of 6 hours.

FIG. 39 is a graph of a rate distance measurement, in plan view, for a time interval of 12 hours.

FIG. 40 is a graph of a rate distance measurement, in plan view, for a time interval of 18 hours.

FIG. 41 is a graph of a rate distance measurement, in plan view, for a time interval of 24 hours.

FIG. 42 is a graph of a solution objective measure for a given source location on a grid per 6 hour elapsed time.

FIG. 43 is a graph of a solution objective measure for a given source location on a grid per 12 hour elapsed time.

FIG. 44 is a graph of a solution objective measure for a given source location on a grid per 18 hour elapsed time.

FIG. 45 is a graph of a solution objective measure for a given source location on a grid per 24 hour elapsed time.

FIG. 46 is a graph of a generated record count for a given source location on a grid in plan view at a 6 hour time interval.

FIG. 47 is a graph of a generated record count for a given source location on a grid in plan view at a 12 hour time interval.

FIG. 48 is a graph of a generated record count for a given source location on a grid in plan view at a 18 hour time interval.

FIG. 49 is a graph of a generated record count for a given source location on a grid in plan view at a 24 hour time interval.

FIG. 50 is a graph of a leak detection coverage map for a spatial distance coverage at 6 hours.

FIG. 51 is a graph of a leak detection coverage map for a spatial distance coverage at 12 hours.

FIG. 52 is a graph of a leak detection coverage map for a spatial distance coverage at 18 hours.

FIG. 53 is a graph of a leak detection coverage map for a spatial distance coverage at 24 hours.

FIG. 54 illustrates wind realizations for a site, wherein the top most portion of the figure shows wind direction, the middle portion of the figure illustrates wind speed, and the bottom portion of the figure illustrates solar radiation changes over time.

FIG. 55 illustrates a second wind realization for the site wherein the top most portion of the figure shows wind direction, the middle portion of the figure illustrates wind speed, and the bottom portion of the figure illustrates solar radiation changes over time.

FIG. 56 illustrates a third wind realization for the site wherein the top most portion of the figure shows wind direction, the middle portion of the figure illustrates wind speed, and the bottom portion of the figure illustrates solar radiation changes over time.

FIG. 57 illustrates another wind realization for the site wherein the top most portion of the figure shows wind direction, the middle portion of the figure illustrates wind speed, and the bottom portion of the figure illustrates solar radiation changes over time.

FIG. 58 illustrates another wind realization for the site wherein the top most portion of the figure shows wind direction, the middle portion of the figure illustrates wind speed, and the bottom portion of the figure illustrates solar radiation changes over time.

FIG. 59 illustrates another wind realization for the site wherein the top most portion of the figure shows wind direction, the middle portion of the figure illustrates wind speed, and the bottom portion of the figure illustrates solar radiation changes over time.

FIG. 60 illustrates another wind realization for the site wherein the top most portion of the figure shows wind direction, the middle portion of the figure illustrates wind speed, and the bottom portion of the figure illustrates solar radiation changes over time.

FIG. 61 illustrates four sensor designs with a fixed design and optimized solution.

FIG. 62 illustrates a six sensor design and optimized solution for the site.

FIG. 63 illustrates search and evaluation grids in one example embodiment of the disclosure.

FIG. 64 illustrates good coverage maximization for one sensor. The first plot shows the optimization profile.

FIG. 65 illustrates a good coverage maximization for one sensor. The second plot shows the site sensor layout.

FIG. 66 illustrates good coverage maximization for two. The first plot shows the optimization profile

FIG. 67 illustrates good coverage maximization for two sensors. The second plot shows the site sensor layout.

FIG. 68 illustrates good coverage maximization for three sensors The first plot shows the optimization profile

FIG. 69 illustrates good coverage maximization for three sensors. The second plot shows the site sensor layout.

FIG. 70 illustrates good coverage maximization for four sensors. The first plot shows the optimization profile

FIG. 71 illustrates good coverage maximization for four sensors. The second plot shows the site sensor layout.

FIG. 72 illustrates good coverage maximization an for five sensors. The first plot shows the optimization profile

FIG. 73 illustrates good coverage maximization for five sensors. The second plot shows the site sensor layout.

FIG. 74 illustrates good coverage maximization for six sensors. The first plot shows the optimization profile.

FIG. 75 illustrates good coverage maximization for six sensors. The second plot shows the site sensor layout.

FIG. 76 illustrates a summary plot of cases.

FIG. 77 illustrates an example method in accordance with one example embodiment of the disclosure for detection and coverage map generation.

To facilitate understanding, identical reference numerals have been used, where possible, to designate identical elements that are common to the figures (“FIGS”). It is contemplated that elements disclosed in one embodiment may be beneficially utilized on other embodiments without specific recitation.

### DETAILED DESCRIPTION

In the following, reference is made to embodiments of the disclosure. It should be understood; however, that the disclosure is not limited to specific described embodiments. Instead, any combination of the following features and elements, whether related to different embodiments or not, is contemplated to implement and practice the disclosure. Furthermore, although embodiments of the disclosure may achieve advantages over other possible solutions and/or over the prior art, whether or not a particular advantage is achieved by a given embodiment is not limiting of the disclosure. Thus, the following aspects, features, embodiments and advantages are merely illustrative and are not considered elements or limitations of the claims except where explicitly recited in a claim. Likewise, reference to “the disclosure” shall not be construed as a generalization of inventive subject matter disclosed herein and should not be considered to be an element or limitation of the claims except where explicitly recited in a claim.

Although the terms first, second, third, etc., may be used herein to describe various elements, components, regions, layers and/or sections, these elements, components, regions, layers and/or sections should not be limited by these terms. These terms may be only used to distinguish one element, components, region, layer or section from another region, layer or section. Terms such as “first”, “second” and other numerical terms, when used herein, do not imply a sequence or order unless clearly indicated by could be termed a second element, component, region, layer or section without departing from the teachings of the example embodiments.

When an element or layer is referred to as being “on,” “engaged to,” “connected to,” or “coupled to” another element or layer, it may be directly on, engaged, connected, coupled to the other element or layer, or interleaving elements or layers may be present. In contrast, when an element is referred to as being “directly on,” “directly engaged to,” “directly connected to,” or “directly coupled to” another element or layer, there may be no interleaving elements or layers present. Other words used to describe the relationship between elements should be interpreted in a like fashion. As used herein, the term “and/or” includes any and all combinations of one or more of the associated listed terms.

Some embodiments will now be described with reference to the figures. Like elements in the various figures will be referenced with like numbers for consistency. In the following description, numerous details are set forth to provide an understanding of various embodiments and/or features. It will be understood, however, by those skilled in the art, that some embodiments may be practiced without many of these details, and that numerous variations or modifications from the described embodiments are possible. As used herein, the terms “above” and “below”, “up” and “down”, “upper” and “lower”, “upwardly” and “downwardly”, and other like terms indicating relative positions above or below a given point are used in this description to more clearly describe certain embodiments.

Aspects of the disclosure concern a method to establish a detectable leak source location (or coverage) map for methane given the prevailing wind conditions encountered, and the known locations and characteristics of the deployed methane sensors. While described as being applicable to methane, other types of contaminants and sensors may be used. There can be two potential modalities of the map, one driven by a synthetic wind generation procedure and the other based on actual wind measurements collected on-site.

Either modality serves to indicate the regions from which an originating leak could be detected for the given wind conditions and detector locations; or correspondingly, the regions with no coverage, such that if a leak had originated in that region, it would not have been detected by the existing sensor arrangement and noted wind conditions, either generated or experienced.

In the first modality, a predictive wind model can be generated for any desired period of time to demonstrate the evolution of the coverage map. In the second modality, the coverage map will evolve in a temporal sense as more wind data and dispersant concentration data is gathered. The computation that generates it can be adjusted to display results periodically, or over various time periods, for example, the previous 24 or 48 hours, or other time window in the past with available wind and sensor data.

Historical wind data or conditioned synthetic data can be used to generate hypothetical maps that can also be used for optimal sensor placement in a planning phase. The coverage map can be overplotted on the facility map to ensure that critical leak-prone elements such as storage tanks, compressors, large valves, etc, are within the covered region. If they are not, changes to sensor placement may be desirable.

In practice, real-world sensor data is replaced by information from the valid forward model (e.g., the Gaussian plume model or more advanced models) to compute the sensor readings for a given leak source. A set of grid points can be defined a priori (over a grid or based on site information comprising known equipment) which can be used as artificial leak source locations. The identification of the stipulated leak source with the prevailing wind conditions will mark that location as either attainable or not. Evaluation of all points in the set will thus lead to a leak source coverage map or feasible detection map. This exercise may be repeated with time giving a temporal evolution of the map. Representative figures are presented below.

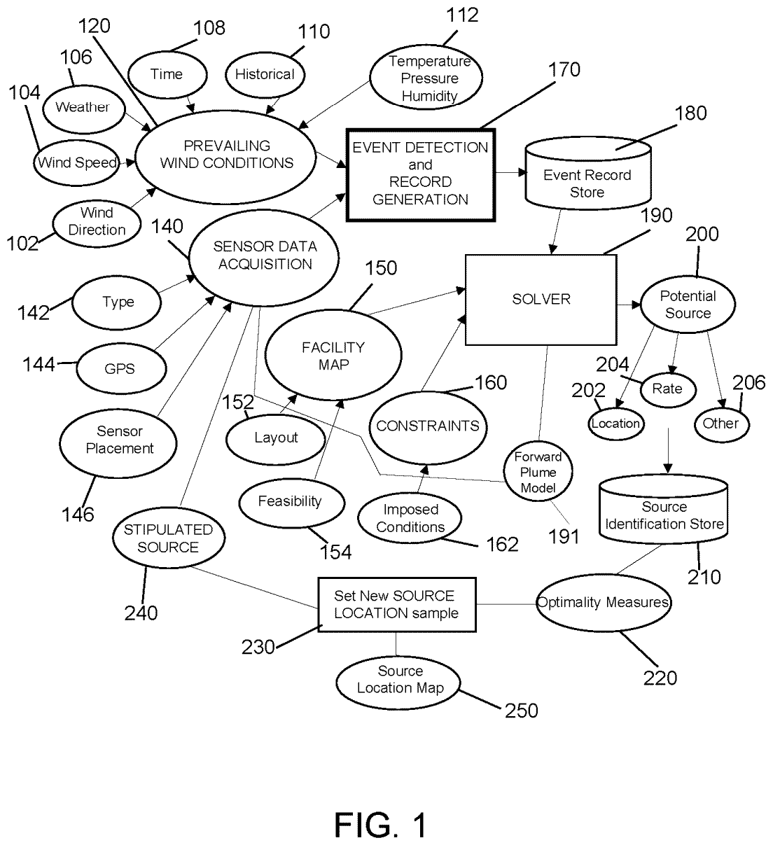

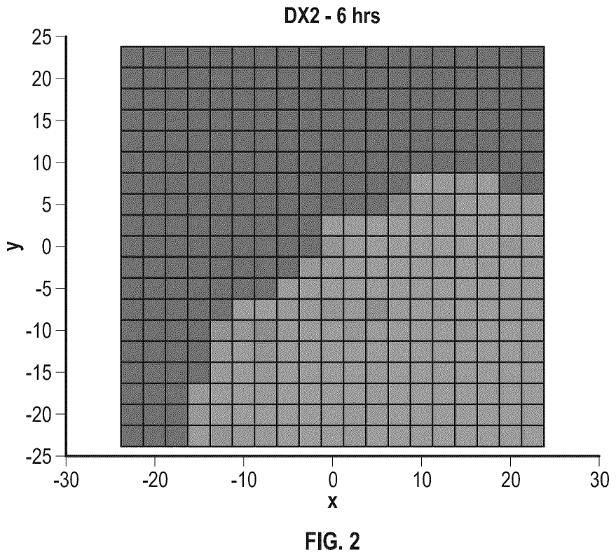

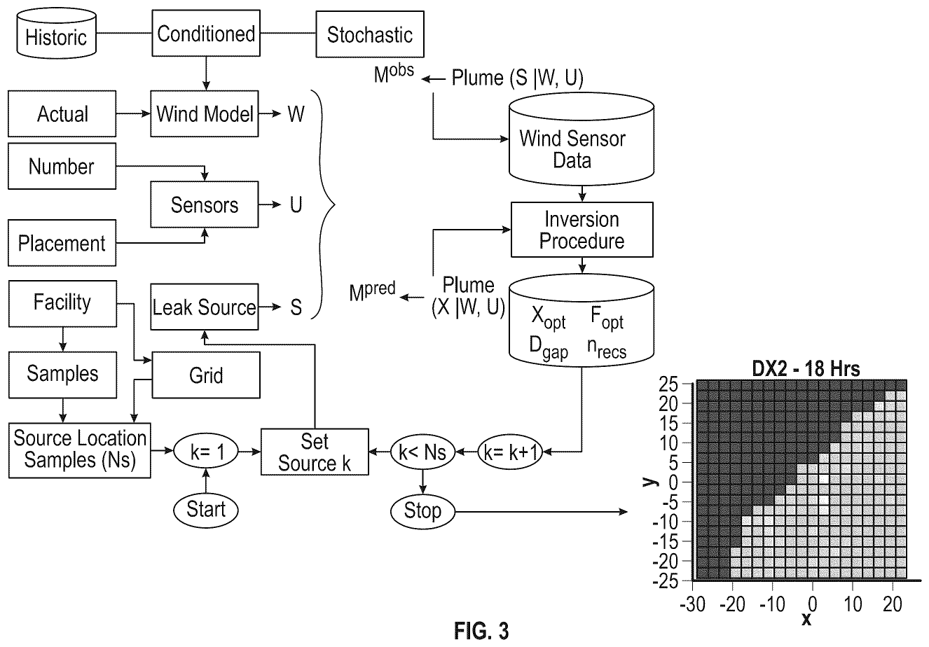

The flowchart in FIG. 1 captures the salient elements of the procedure developed for methane sensor inversion. These include a wind model, a forward plume model, and an inversion engine. It is necessary only to appreciate that the wind model may be conditioned based on historical data (as a function of the time of year) and that a synthetic wind model may be used to place the sensors. In practice, real wind data gathered from an on-site anemometer may be used. The sensor data is then derived from anticipated leak source location and the prevailing wind conditions. As shown in FIG. 1, the inversion procedure (if applicable with an appropriate number of records) will return a source location. As the source is given for each test, a distance measure can be used as a measure of inversion quality. The process may thus repeat, for all leak source points in the set, giving rise to a map as shown in FIG. 2. The procedure will then repeat periodically with consideration of new wind conditions. The schema is given in FIG. 3.

Referring to FIG. 1, the wind conditions are convolved with the sensor data that is generated using the forward Gaussian plume model for record generation. This information is used along with facility specific constraints to solve the source location problem. An optimality measure can be estimated as the source is known for each sample point tested, ultimately, leading to a feasible location map, as per FIG. 2, and a leak source detection schema, as per FIG. 3.

Different inputs may be provided to create the prevailing wind conditions 120. These inputs may be wind direction 102, wind speed 104, weather 106, time 108, historical data 110 and temperature, pressure and humidity 112. These are provided into a record and sent to event detection and record generation 170. In addition to this data, sensor data may be gathered. Types of sensors 142, global positioning system (GPS) locations 144, and sensor placement details 146 are gathered and provided to the sensor data acquisition 140 and eventually to the event detection and record generation 170. The event detection and record generation processes this data and organizes it to provide to an event record store 180.

A geographical layout 152 and feasibility analysis 154 are provided to create a facility map 150 which, along with the event record store 180 input to the solver 190. Imposed conditions 162 may be fed into a constraints section 160 that are fed into the solver 190. A forward plume model 191 may also be created and connected with the solver 190 and the sensor data acquisition 140. The solver 190 is created to process this data and develop a potential source 200. The potential source 200 may have a location 202, rate 202 and other data 206. Data from the potential source 200 may be stored in a source identification store 210. In addition to the source identification store, optimality measures 220 may be identified. Block 230 sets a new source location. This becomes the stipulated source in 240, that is an input to the sensor data acquisition block 140. The optimality measures 220 are applied to each identified source added in block 210. If there are no new source locations to consider in 230, the coverage map is returned in 250.

Negative values indicate no solution, while high values indicate poor inversion result. Distance values (or optimality gap) close to zero indicate closeness to the stipulated leak source. This indicates the regions where an actual source may be more readily identified. Note that in practice, where the variability in ideal wind conditions may limit the quality of the results, the map is a useful guide (given knowledge of the facility layout and equipment therein).

An example embodiment of the disclosure is presented. In this disclosure, a temporal evolution of a methane sensor-inversion process for given wind conditions is presented. In this embodiment, the solution varies over a grid or set of possible leak sources given one, or more, realizations of a synthetic wind model with a fixed set of methane sensors. In one embodiment, the method permits a method in which the prevailing wind conditions, actual or simulated, together with a set of predefined leak sources, can indicate the utility of the inversion procedure. As will be understood, a grid over the site (as demonstrated) may be used or a pre-defined set of points over the area of interest may also be used. These points can vary in location (x,y), height (z) and indeed, the leak rate r. Additionally, a coverage map may be used for different leak rates.

In one embodiment, the output or outcome of this procedure is a detection coverage map that will evolve with time as new-wind sensor data is gathered.

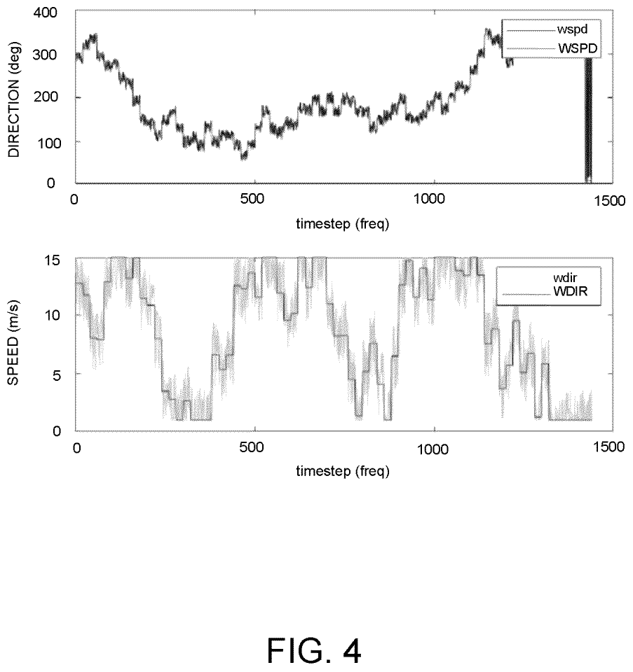

In the example embodiment, a wind model is used to generate a wind profile over a given selection of time. In this embodiment, a period of 24 hours is chosen. As part of the data, the temporal wind direction and speed are shown in FIG. 4.

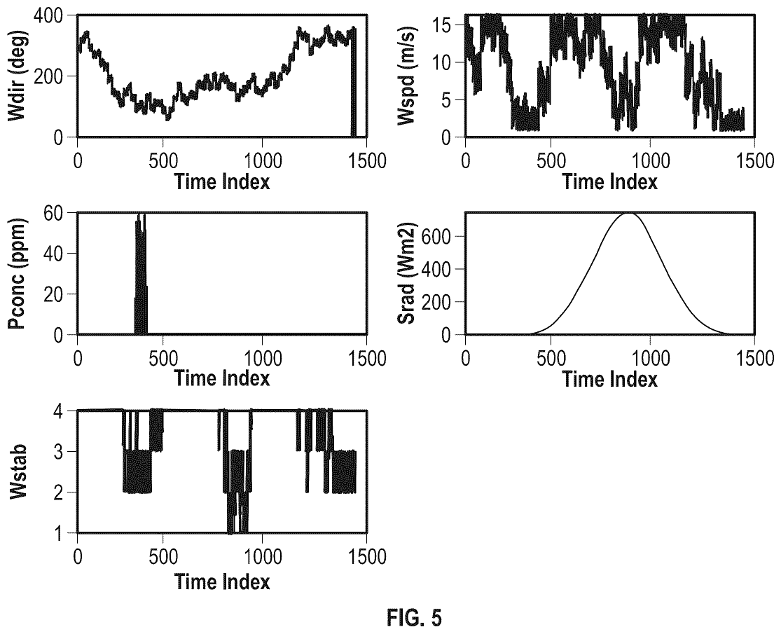

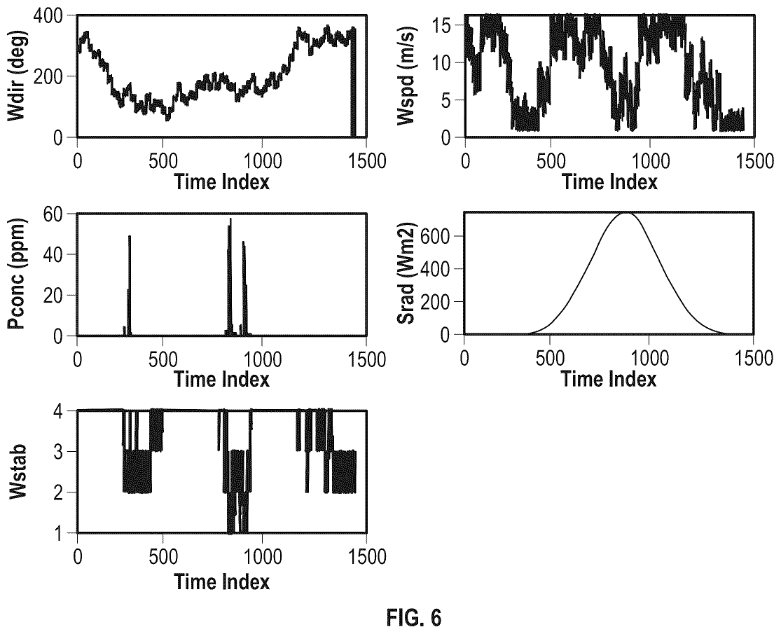

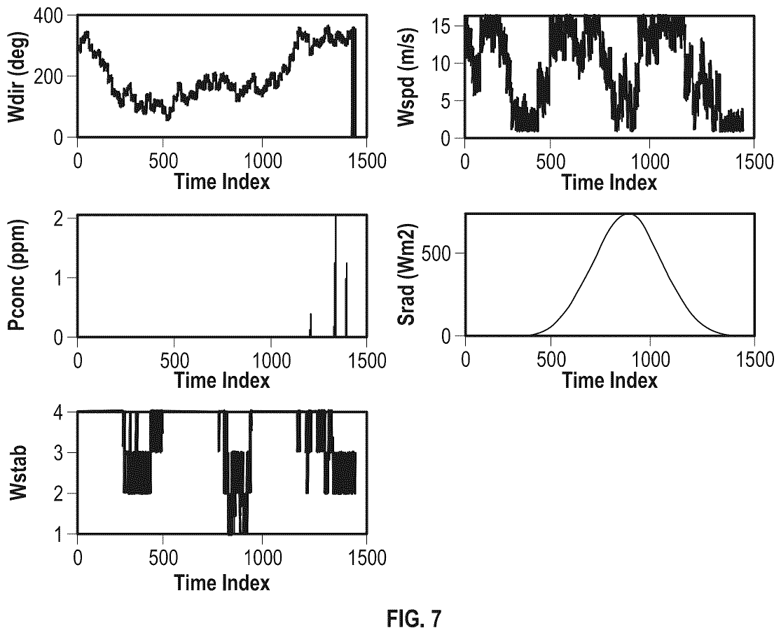

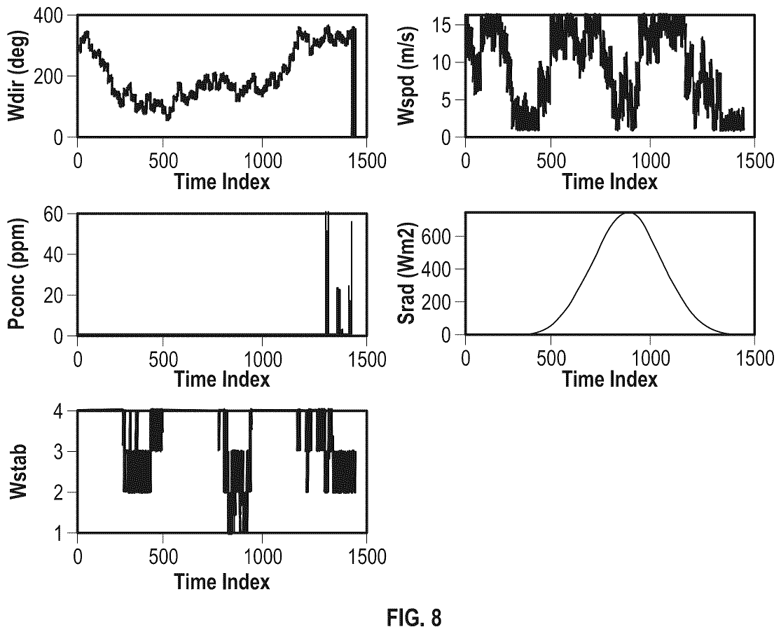

In this example, four sensors are arranged in a plus-sign pattern on a pad. The sensor data gathered at each location is shown in FIGS. 5 to 8. Each plot comprises sub plots. These sub plots show wind direction (in degrees), wind speed (in meters/second), concentration reading (in ppm), solar radiation (W/m2) and inferred wind stability by index. In these embodiments, a Gaussian plume model is used as the forward model to establish the anticipated methane concentration. As will be understood, the Gaussian plume model is but one type of model to be used in forward modeling and should not be considered limiting.

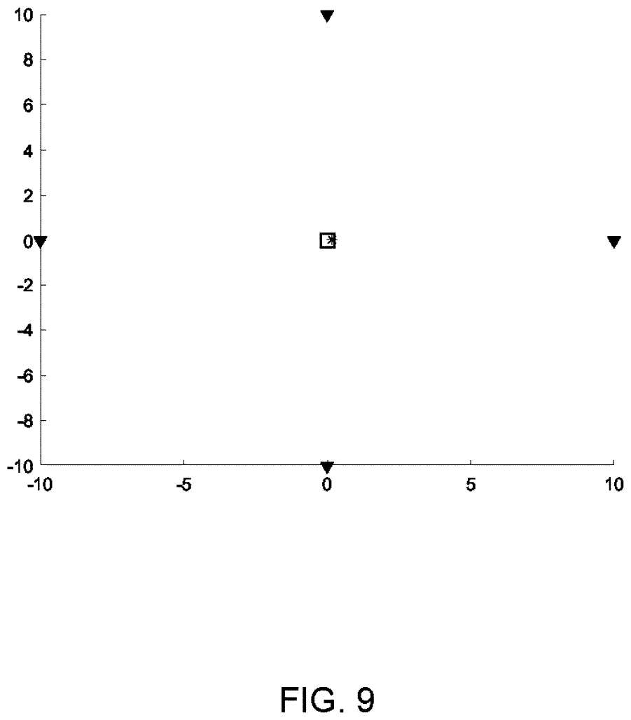

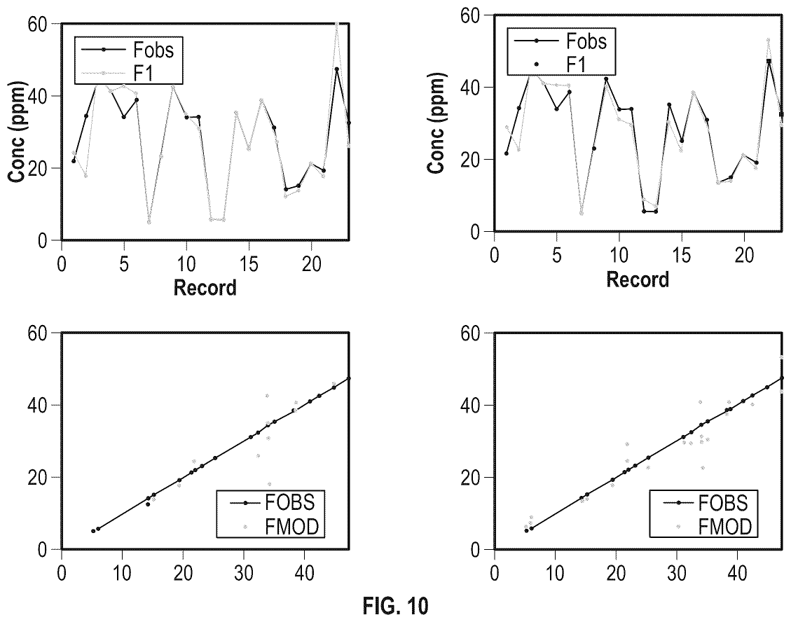

In this embodiment, FIG. 9 shows a layout of 4 sensors (triangles), the known source (square) and the solution from the inversion procedure (star) over the entire period of 24 hours. The objective measure at the known source and the solution are given in FIG. 10.

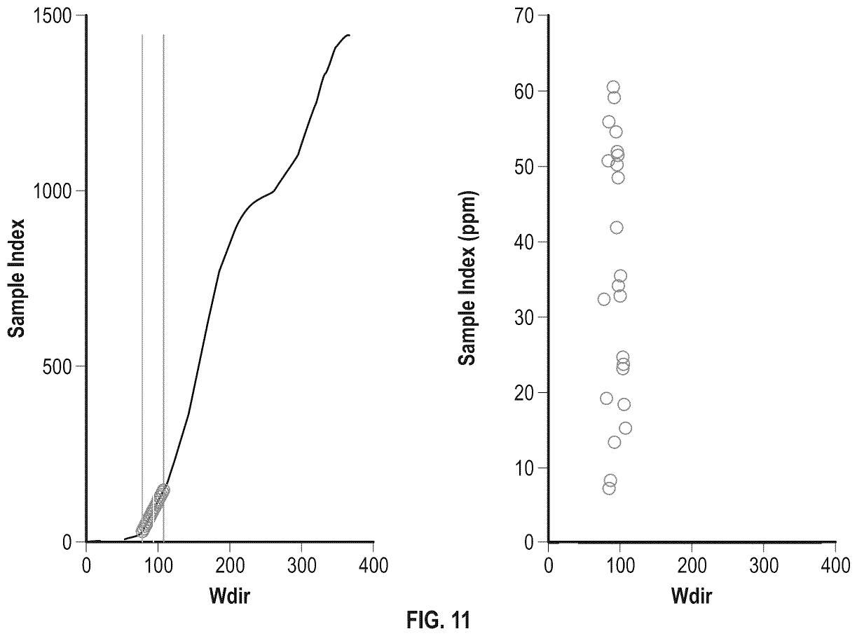

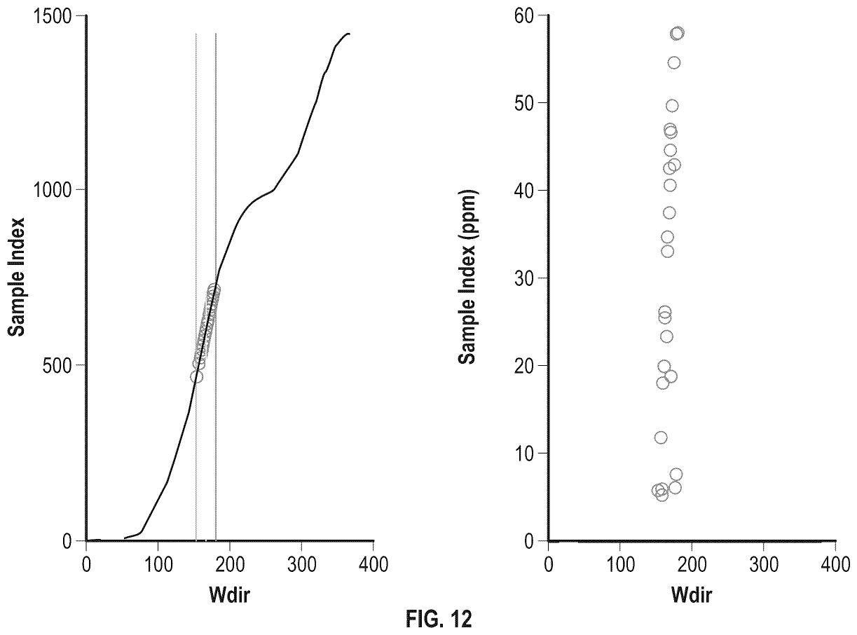

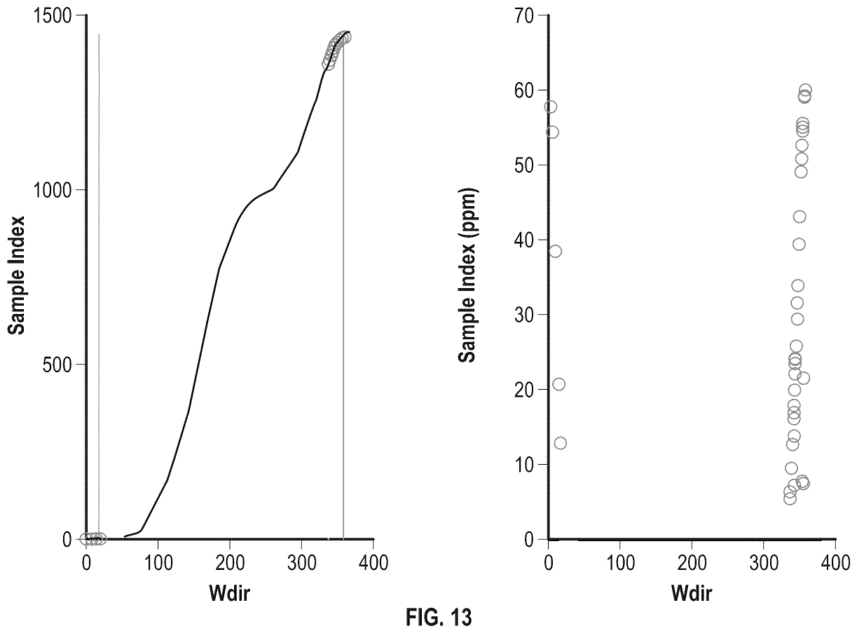

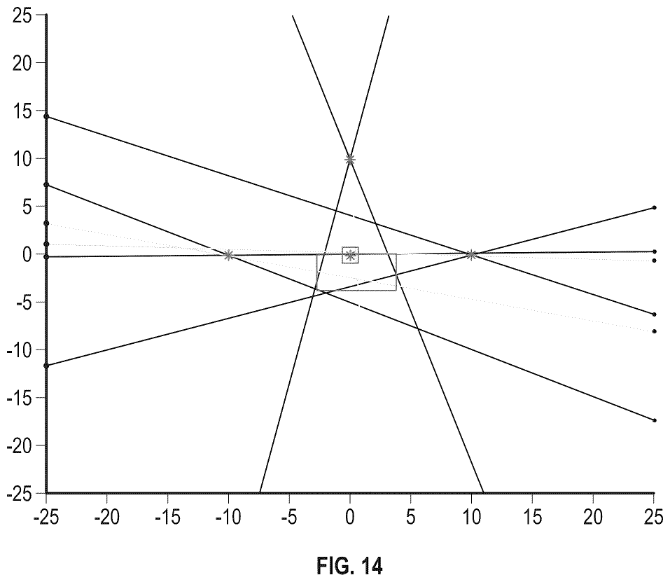

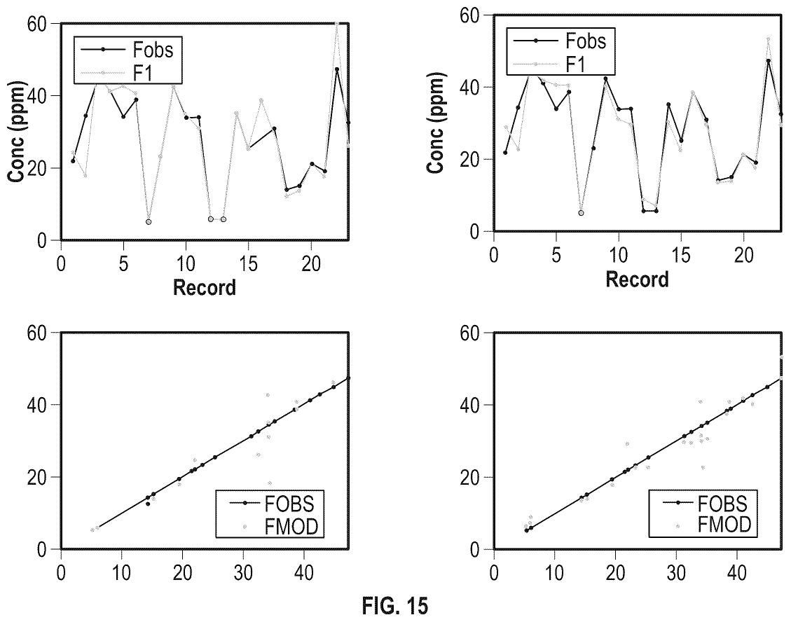

In one embodiment, a cone generation procedure is applied to all sensors (comprising meaningful data) to yield valid linear cuts. In this embodiment, three of the four sensors provide meaningful results that may be used to improve the model. The cone generation plots are shown for FIGS. 11 to 13 for sensors 1, 2 and 4 respectively. The collection of linear cuts are shown in FIG. 14 and the objective measure at the source and solution are given in FIG. 15.

For test purposes, a known source is stipulated at various locations on a pad over a simple 3 by 3 grid. Other sizes of grids may be used. In the following, for each of the nine source locations (starting from bottom left to top right in upward column moves), two plots are provided: the inversion solution with generated cones and the objective measure at source and solution points.

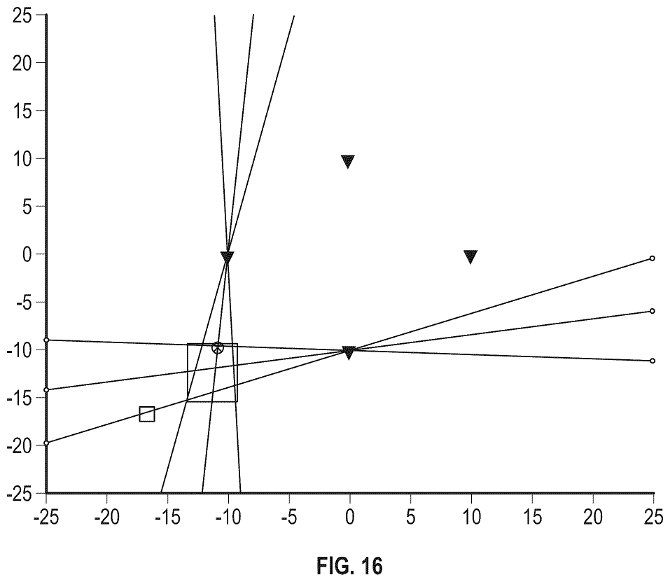

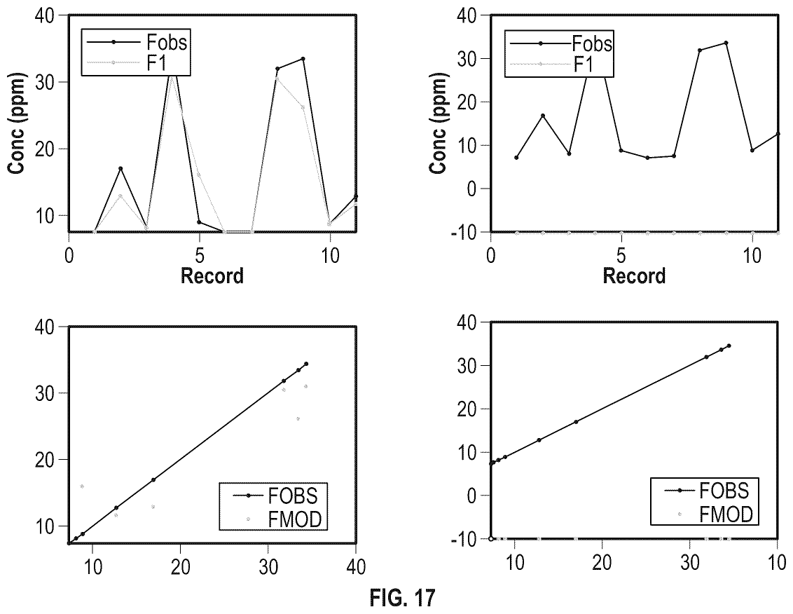

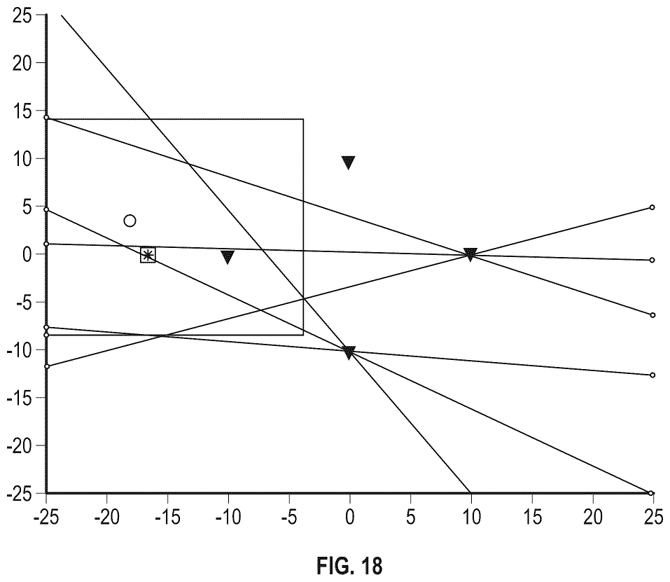

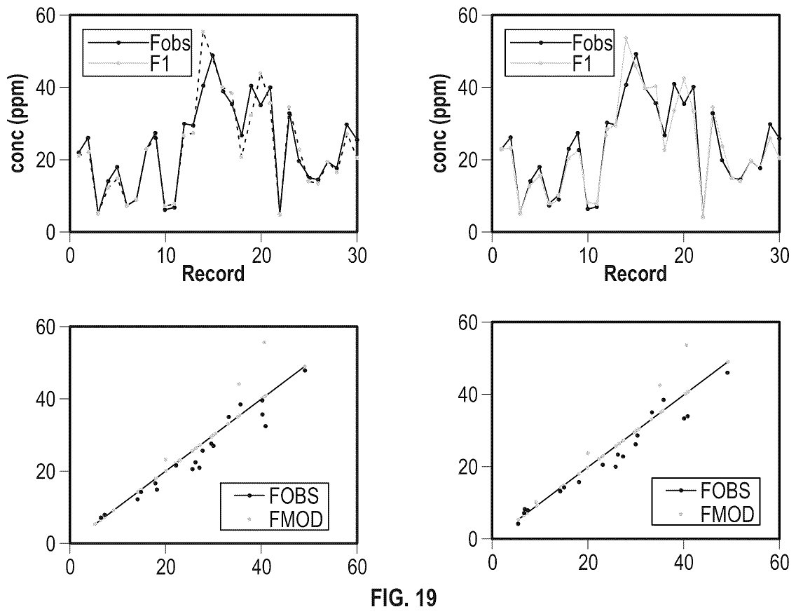

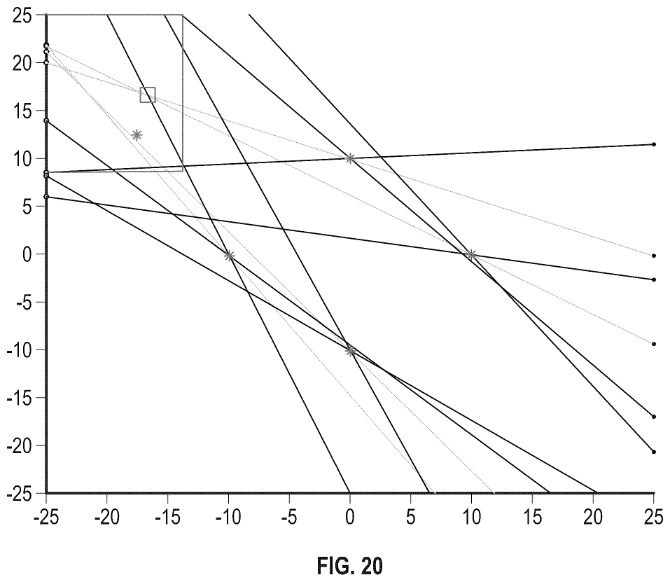

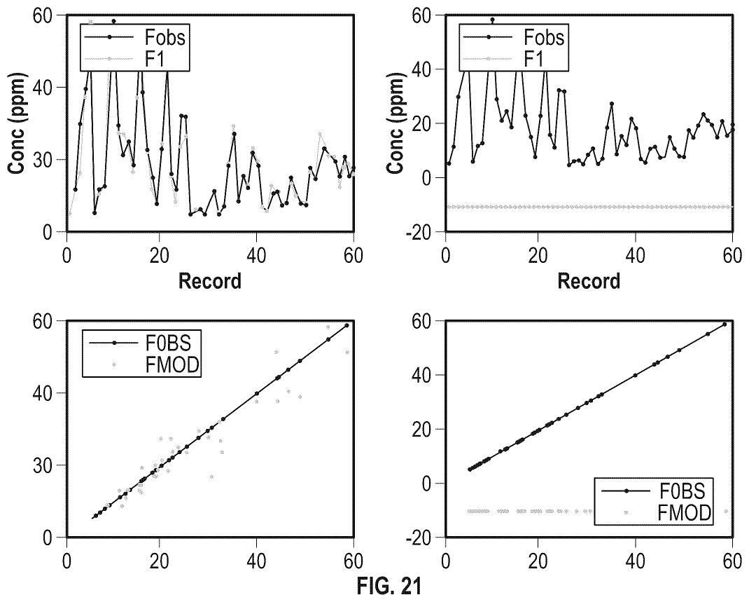

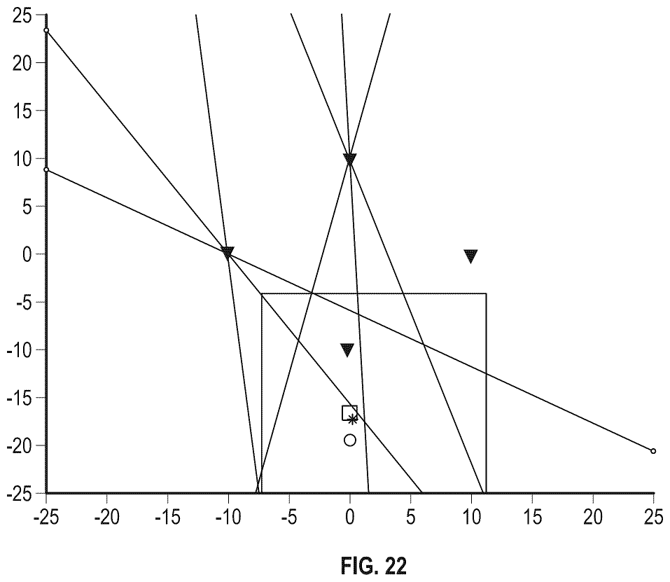

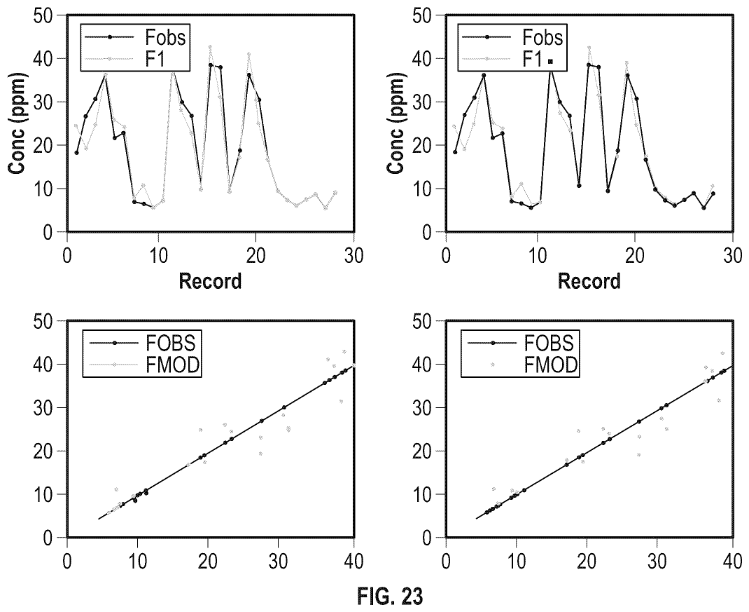

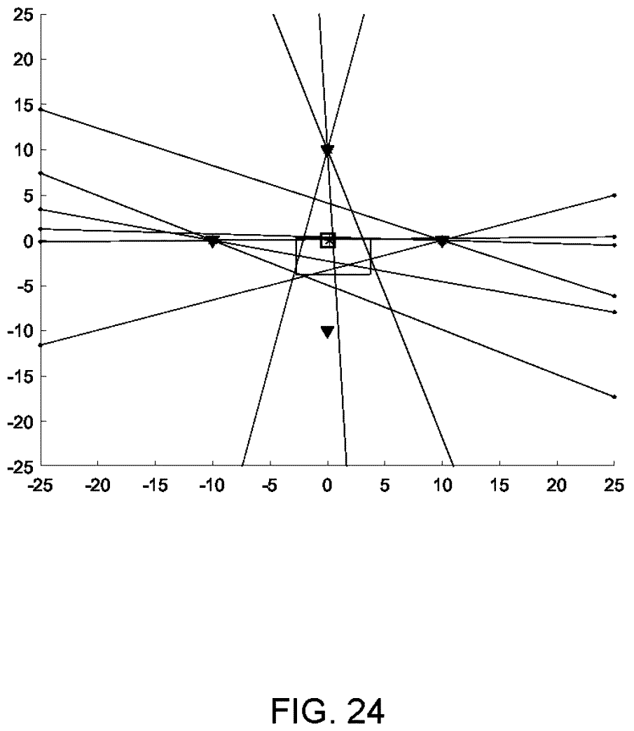

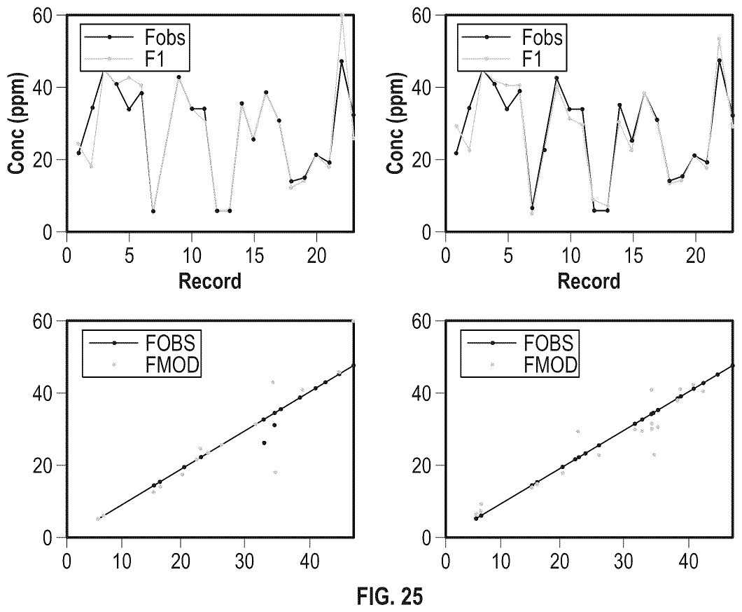

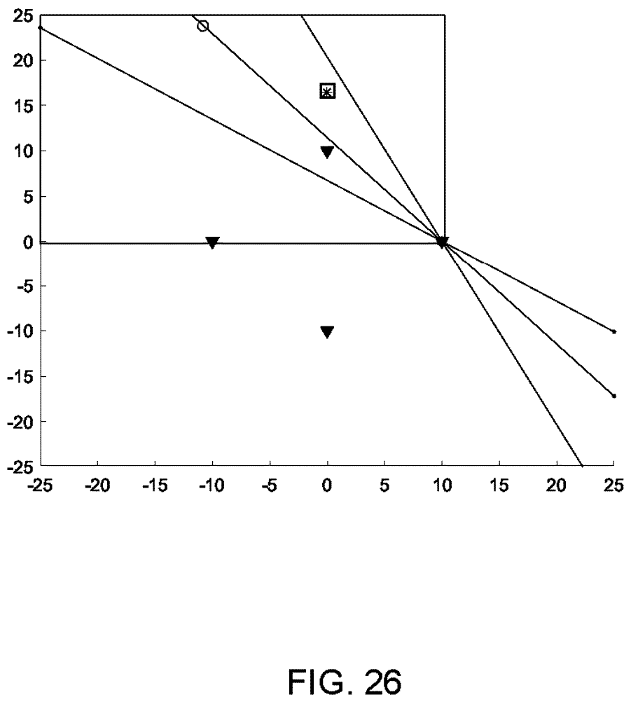

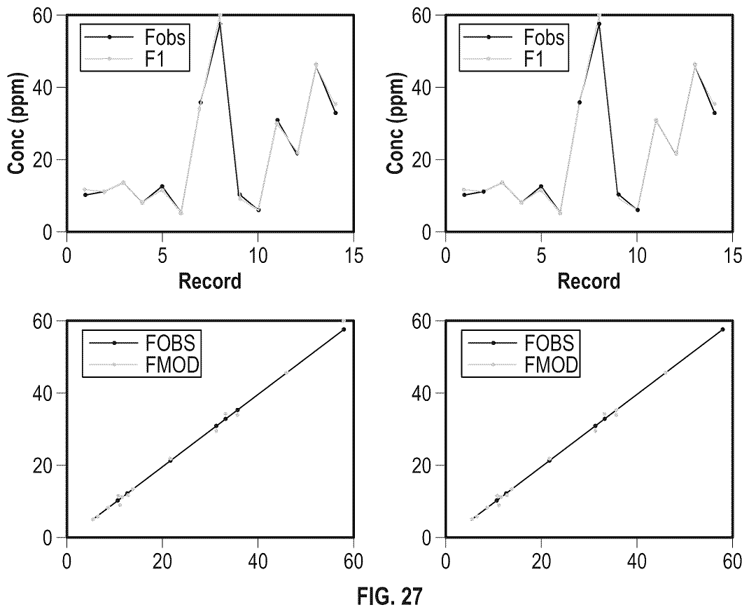

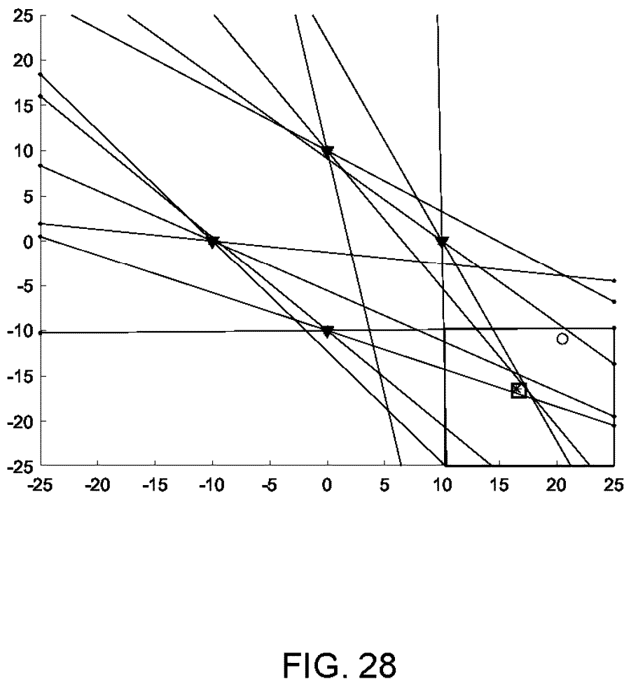

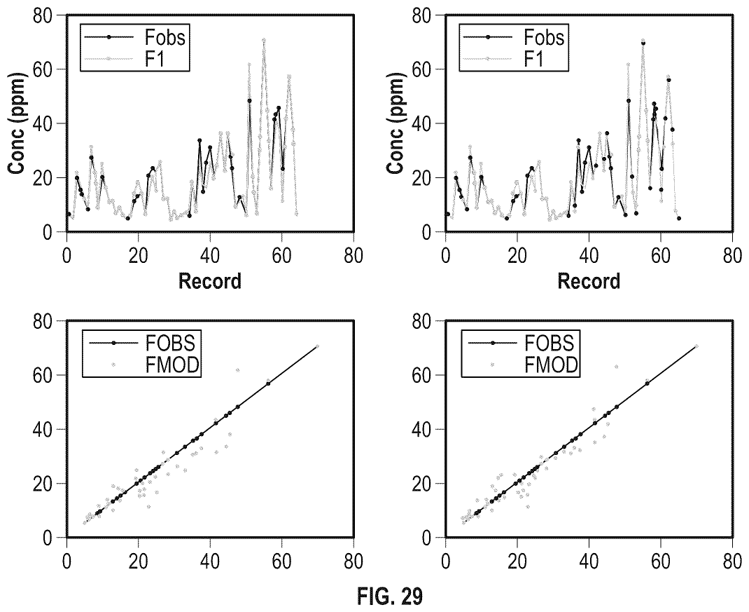

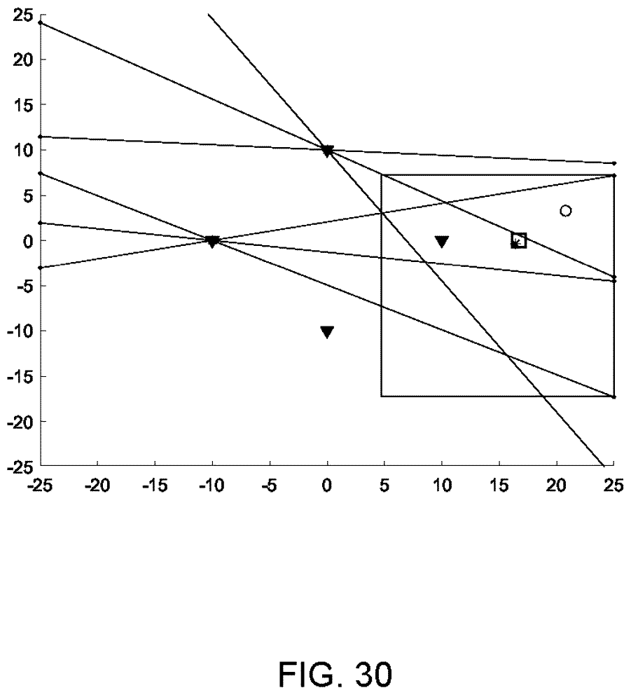

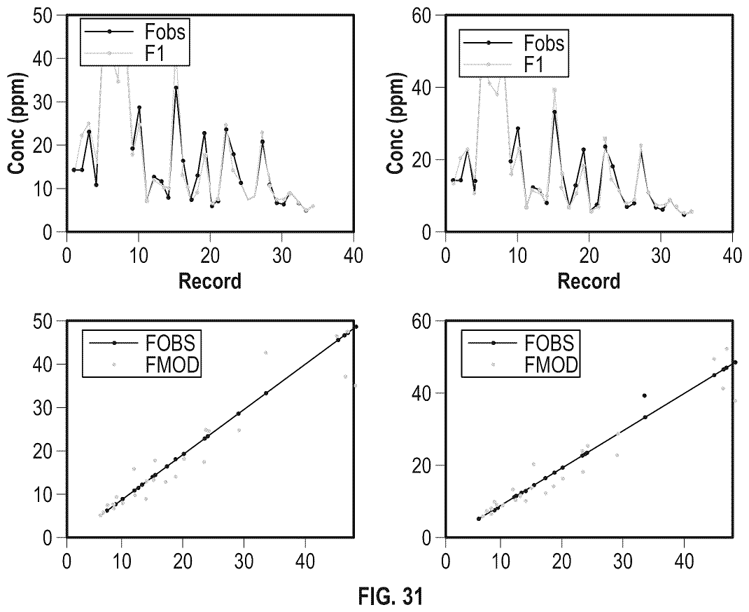

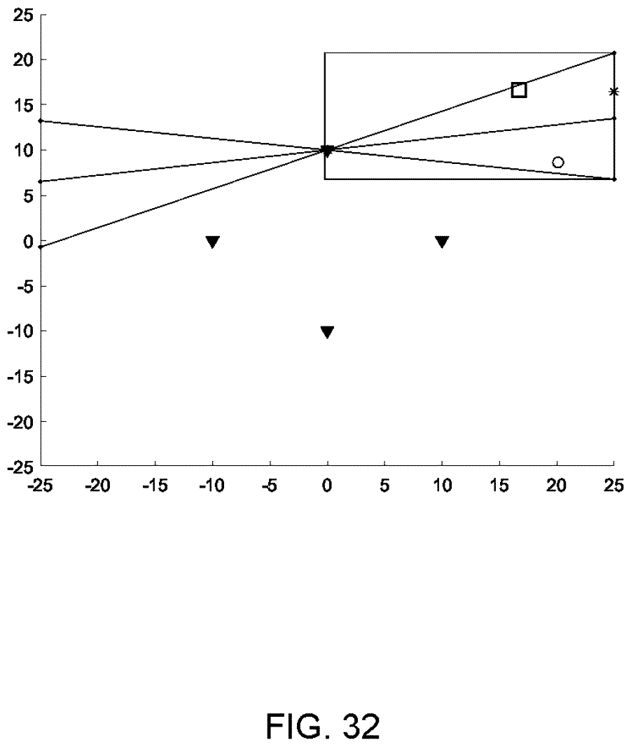

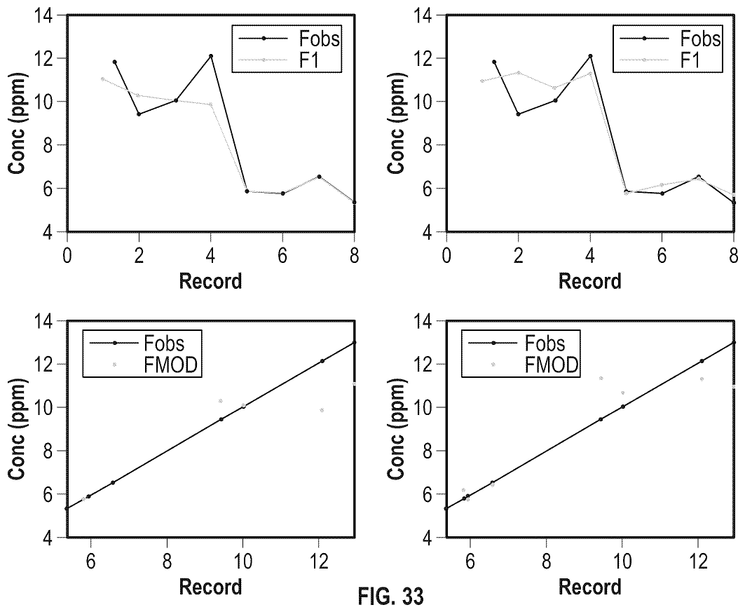

The purpose of these tests is to demonstrate how the inversion procedure accommodates varying positions of a known leak source for a given wind profile over the prevailing period of 24 hours in this example. After this, the temporal evolution of this procedure is demonstrated on a refined grid at intervals of 6, 12, 18 and 24 hours. This indicates the utility of the inversion procedure to a given leak on the grid as a function of wind conditions with time. The set of points evaluated need not be uniformly placed on a grid and could contain the known potential leak source points on the site given the equipment in place. Results are illustrated in FIGS. 16 to 33.

### Temporal Grid Evolution

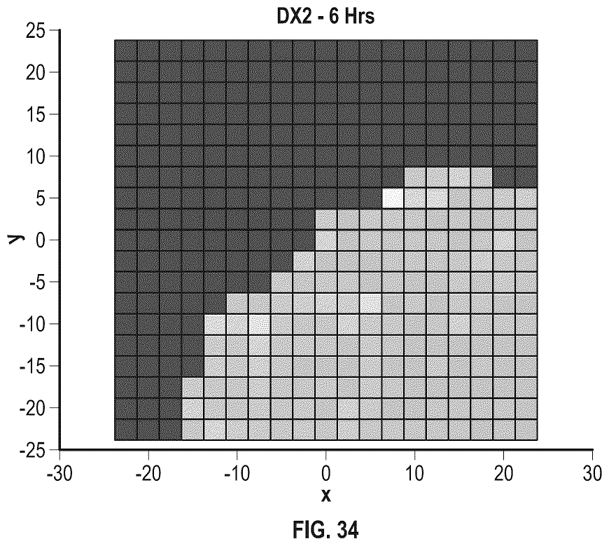

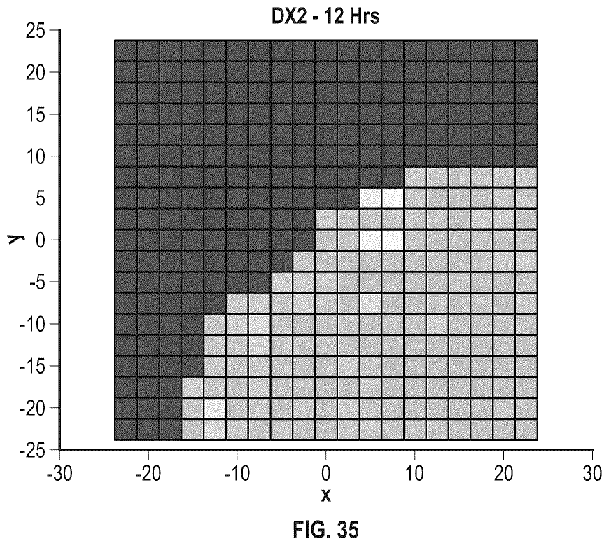

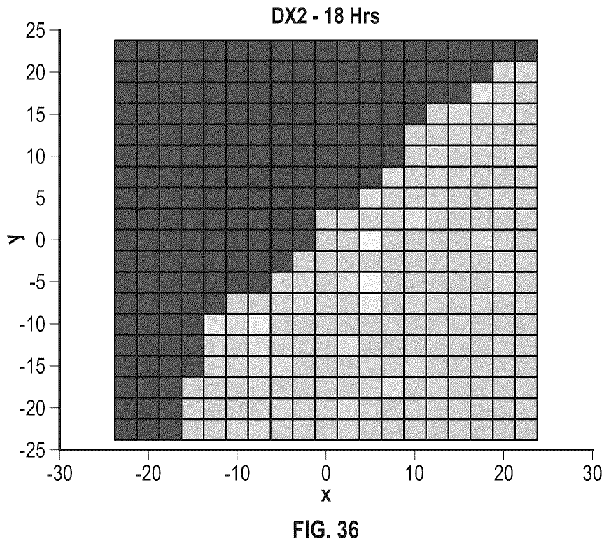

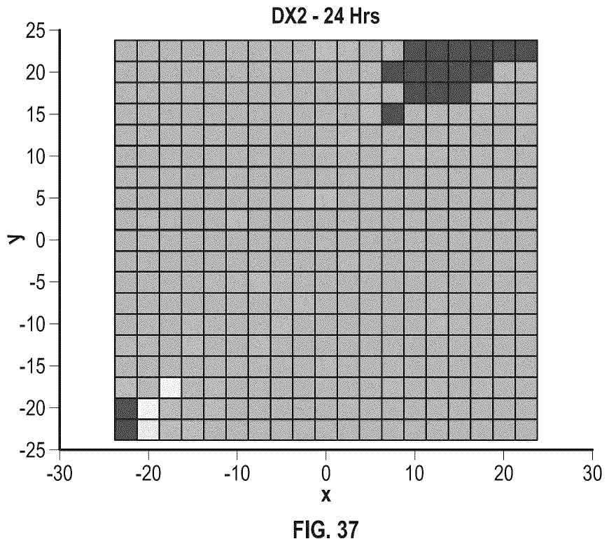

Referring to FIGS. 34 to 37, the optimality gap (over x,y in meters) for the given source location on the grid is shown in plan view at time intervals of 6, 12, 18 and 24 hours respectively. The negative value indicates that no solution was possible due to an insufficient number of records. Other types of FIGS may be generated, including of three (3) space (x,y,z), so the two dimensional plan view should not be considered limiting.

### Rate Distance Measurement

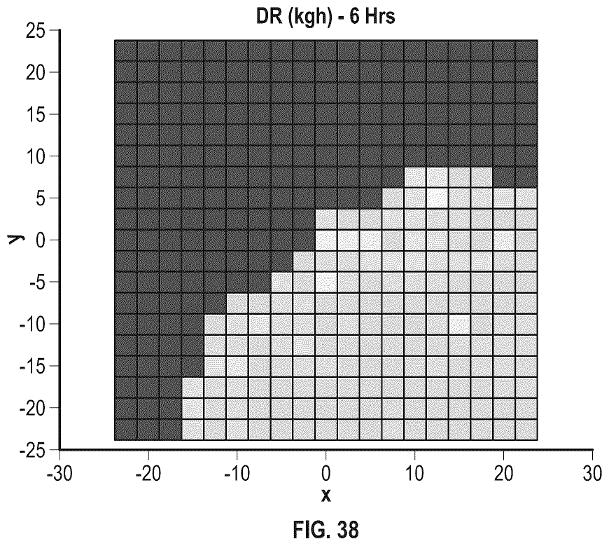

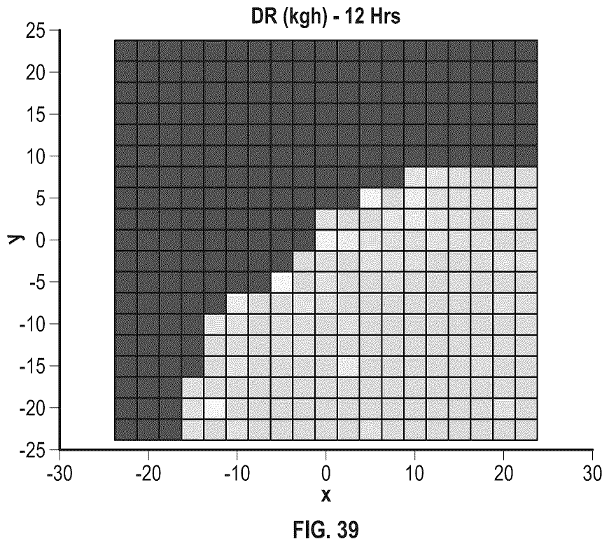

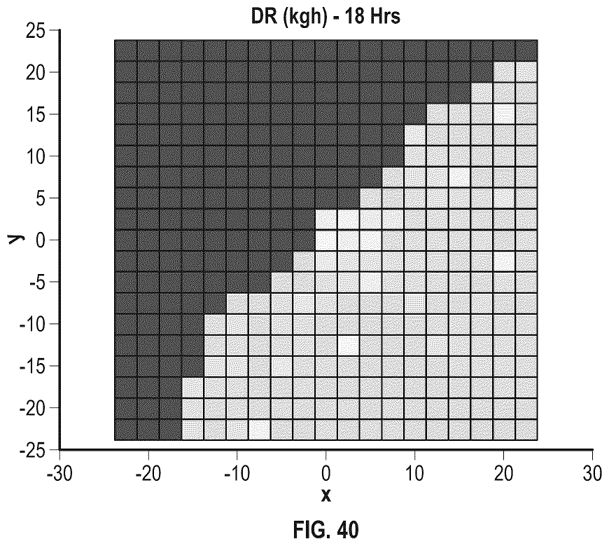

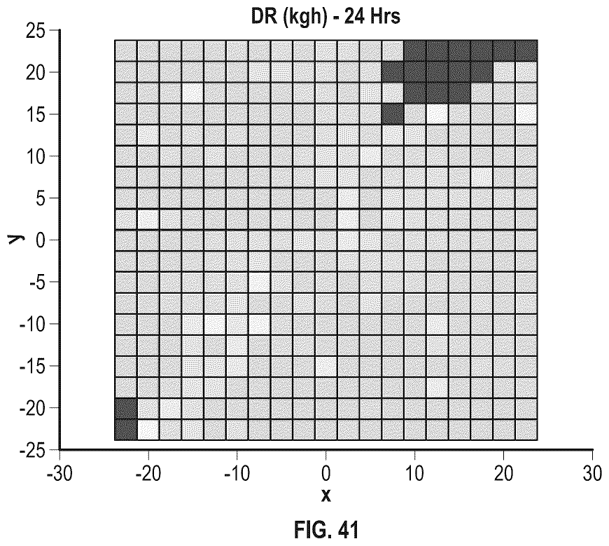

Referring to FIGS. 38 to 41, the optimality gap in the rate (kg/h) for the given source location on the grid is shown in plan view, at time intervals 6, 12, 18 and 24 hours. The negative value indicates that no solution was possible due to an insufficient number of records.

### Objective Measure

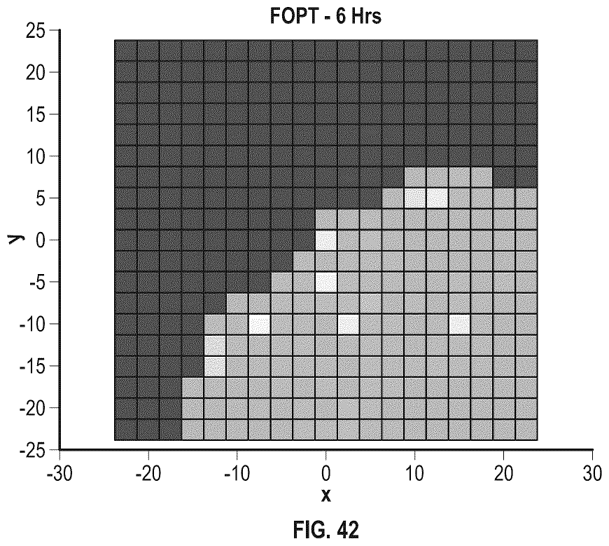

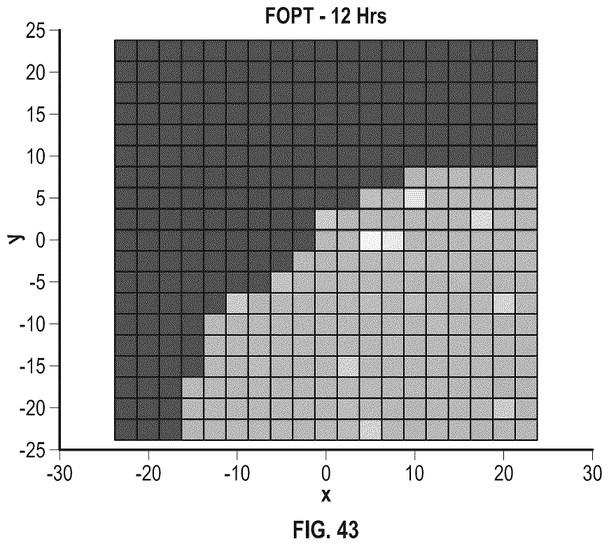

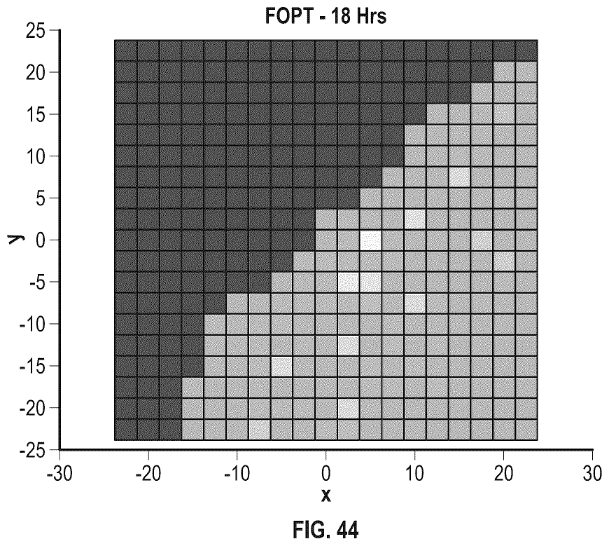

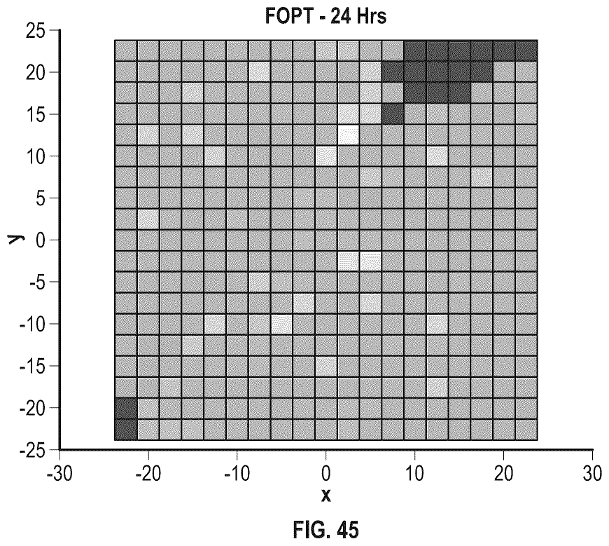

The solution objective measure (Fopt) for the given source location on the grid is shown in plan view in FIGS. 42 to 45 at time intervals 6, 12, 18 and 24 hours respectively. A negative value indicates that no solution was possible due to an insufficient number of records.

### Generated Records

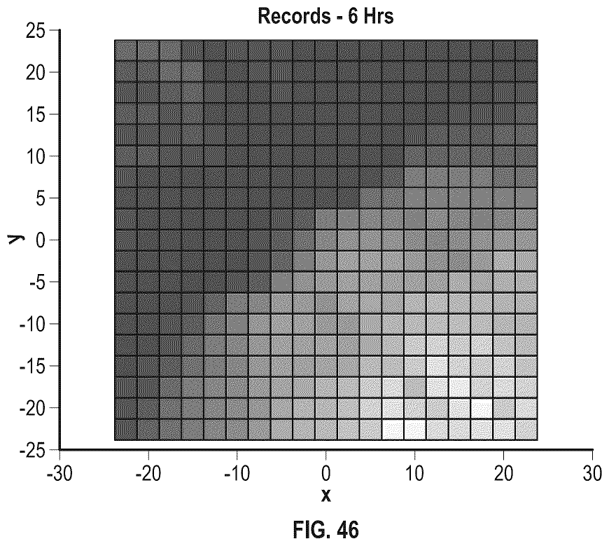

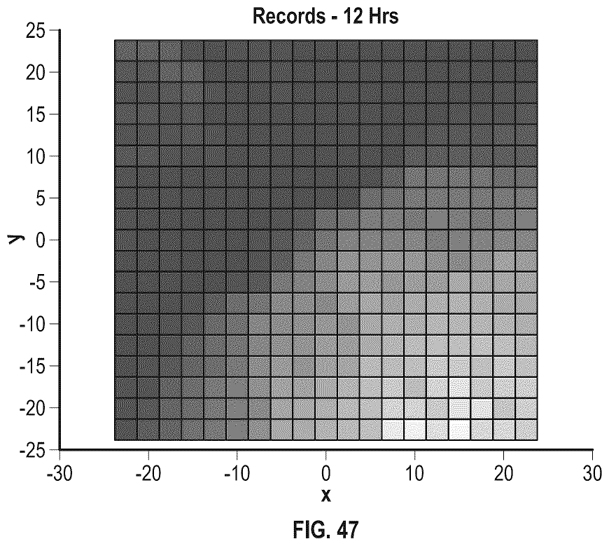

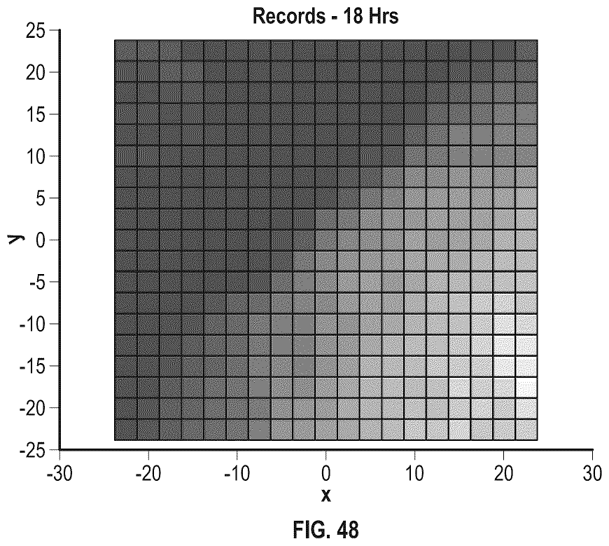

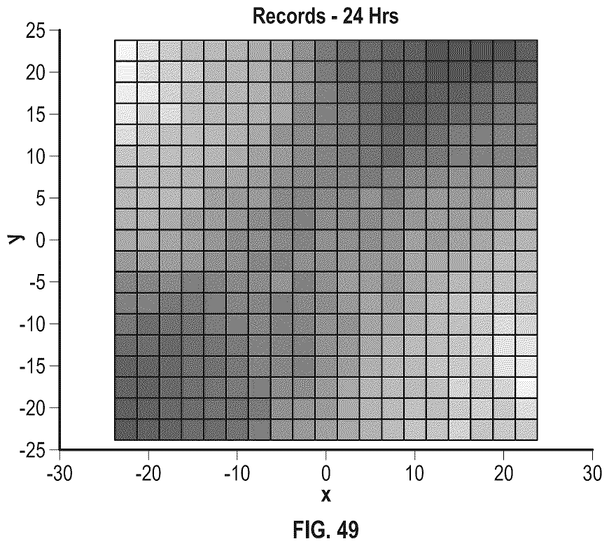

FIGS. 46 to 49 indicate the ‘count of records generated’ according to one example embodiment of the disclosure. As will be understood, the count of generated records are dependent upon the particular parameters input for analysis.

A number of records are illustrated for a given source location on the grid in plan view at time intervals 6, 12, 18 and 24 hours respectively.

### Leak Detection Coverage Maps

In the preceding section, various solution metrics were plotted as surfaces over time. This data can be parsed through a threshold measure to provide leak detection coverage maps for easy estimation of detectability. That is, present a coverage map comprising simple markers, where a first set of markers indicate no possible solution, a second set of markers indicates an acceptable solution and a third set of markers indicates a poor solution was established.

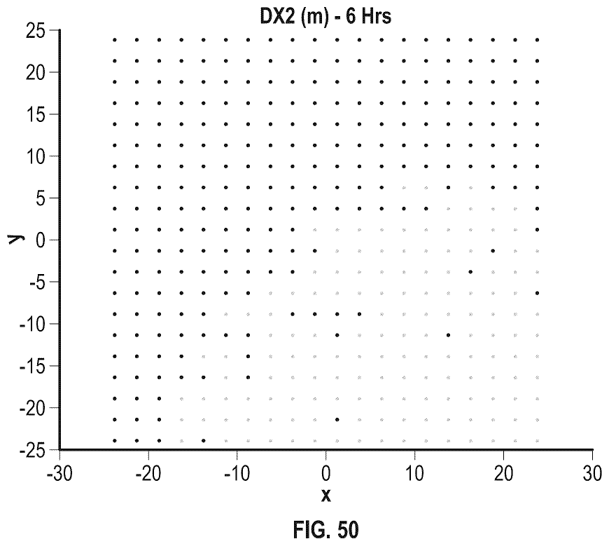

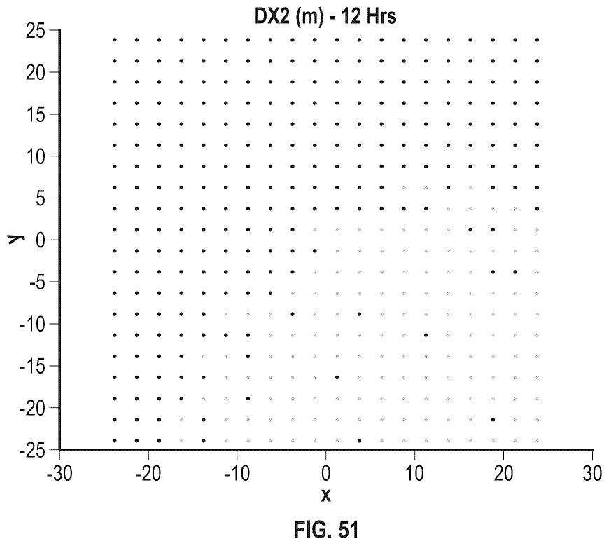

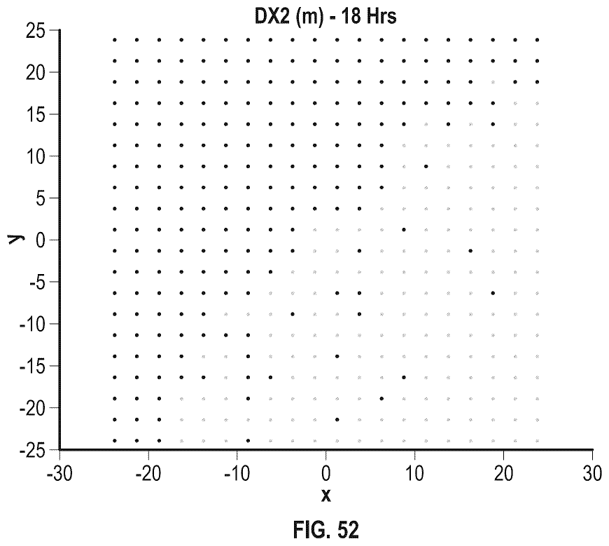

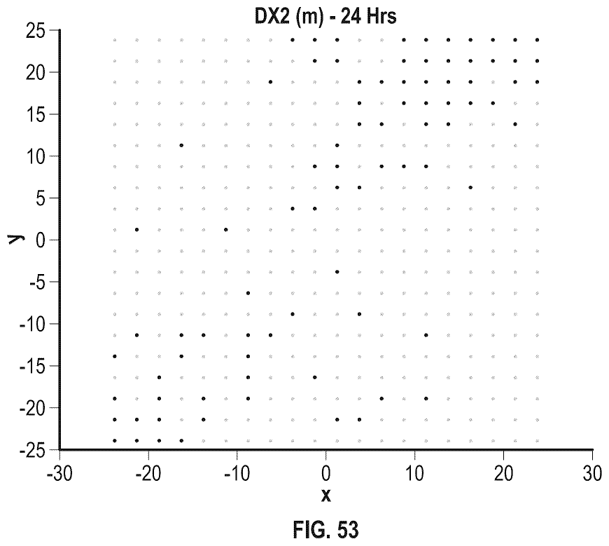

Referring to FIGS. 50 to 53, evolving coverage maps for a distance (D2) at time intervals of 6, 12, 18 and 24 hours are shown.

The results presented in this document demonstrate how a forecast wind model based on a stochastic generation procedure can be used to assess the utility of the sensor inversion method over time. The assumption of a known leak at various points on a grid (or over a collection of sample points indicative of site equipment) can be used to infer if the inversion procedure can correctly identify a potential source. The wind model may be conditioned to historical wind data at the desired location as a function of time in the year.

In practice, the process can be applied in a temporal setting with the acquisition of real wind data to generate a source identification coverage map. That is, to establish if a known source can be located given the prevailing wind conditions. The coverage map can help identify feasibility of source locations that may, or may not, be contributing to the detected reading at the known sensors. Thus, indicating the regions in which a source could reasonably appear for the given conditions and those which are not yet detectable. Complete coverage over time would indicate the source locations that can potentially be identified, and those which cannot. Indication of leaks in the absence of dead zones would thus indicate a no leak situation or fugitive source from elsewhere.

In the given procedure, the sensors are assumed known and fixed. It is clear; however, that the procedure described above can be used to evaluate the value of sensor placement.

### Error Functions

The mathematical model for the inversion problem is stated as follows:

$$min \qquad F ( X ) = \sqrt{\frac{1}{R} {\sum }_{i = 1}^{R} {( {M}_{i}^{obs} - {M}_{i}^{pred} ( X \vert {W}_{i} {U}_{i} ) )}^{2}}$$

$$s . t \qquad G ( X ) \leq 0$$

$${x}_{L} \leq {x}_{i} \leq {x}_{U}$$

$$X ∈ {ℝ}^{n} \qquad ( X ∈ {ℤ}^{n} )$$

$${M}^{pred} = plume ( X \vert W , U )$$

$$X = [ \begin{matrix}{s}_{x} & {s}_{y} & {s}_{z} & {s}_{r}\end{matrix} ]$$

$$W = [ \begin{matrix}{w}_{dir} & {w}_{spd} & {w}_{stab}\end{matrix} ]$$

$$U = [ \begin{matrix}{u}_{x} & {u}_{y} & {u}_{z}\end{matrix} ]$$

$${REC}_{i} = {[ \begin{matrix}W & U & {M}^{obs}\end{matrix} ]}_{i}$$

The error measure F(X) concerns minimization of the sum of residuals from each record in the collection REC of size R. Here, X defines the set of control variables (which includes the source location, rate and possibly, other elements), while W is the wind condition and U is the sensor information associated with each record, with noted observation Mobs. The variables are specified within given bounds, and may be specified as either continuous or discrete depending on need. G(X) defines the set of constraints if valid linear cuts are generated and employed as part of the inversion procedure. The optimal set of control variables and the associated error function value are denoted by Xₒₚₜ and Fₒₚₜ, respectively. The predictive Gaussian plume model, defined as functional plume, returns the model response M^{pred} for the given wind (W) and sensor conditions (U) accordingly.

Let the optimal set of control variable Xₒₚₜ indicate the leak source location (sₓ s_{y} s_{z}) and its rate sᵣ. Similarly, let the known source settings for a given test case be denoted S with elements [s̅ₓ s̅_{y} s̅_{z} s̅ᵣ]. The following optimality test conditions can be defined in order to compare the quality of the solution obtained.

$${D}_{2} = {‖ {X}_{v} , {S}_{r} ‖}_{2}$$

$${X}_{r} = [ \begin{matrix}{s}_{x} & {s}_{y}\end{matrix} ]$$

$${S}_{r} = [ \begin{matrix}{\overline{s}}_{x} & {\overline{s}}_{y}\end{matrix} ]$$

$${D}_{3} = {‖ {X}_{r} , {S}_{r} ‖}_{2}$$

$${X}_{r} = [ \begin{matrix}{s}_{x} & \begin{matrix}{s}_{y} & {s}_{z}\end{matrix}\end{matrix} ]$$

$${S}_{r} = [ \begin{matrix}{\overline{s}}_{x} & \begin{matrix}{\overline{s}}_{y} & {\overline{s}}_{z}\end{matrix}\end{matrix} ]$$

$${D}_{4} = {‖ {X}_{n} , {S}_{n} ‖}_{2}$$

$${X}_{n} = [ \begin{matrix}{s}_{x}^{n} & \begin{matrix}{s}_{y}^{n} & {s}_{z}^{n} & {s}_{r}^{n}\end{matrix}\end{matrix} ]$$

$${S}_{n} = [ \begin{matrix}{\overline{s}}_{x}^{n} & \begin{matrix}{\overline{s}}_{y}^{n} & {\overline{s}}_{z}^{n} & {\overline{s}}_{r}^{n}\end{matrix}\end{matrix} ]$$

$${D}_{r} = {‖ {s}_{r} , {\overline{s}}_{r} ‖}_{2}$$

In the above, the measure D₂ is over the spatial 2D space (x, y) and D₃ is over the spatial 3D space (x, y, z). DA is over the set of all controlled variables including the source rate term. As the scale of the rate (kgh) differs from the spatial scale, the elements should be normalized before comparison, given generically, as Xₙ and Sₙ. Lastly, Dᵣ represents the source rate gap term only. These optimality measures, or other suitable tests, can be used to assess the quality of each solution. D₂ was demonstrated in the figures presented herein (labelled as DX2 in the plots).

Aspects of the disclosure provide an apparatus and methods that are easier to operate and less time consuming than conventional apparatus and methods. In such aspects, considerable advantages exist compared to conventional technologies.

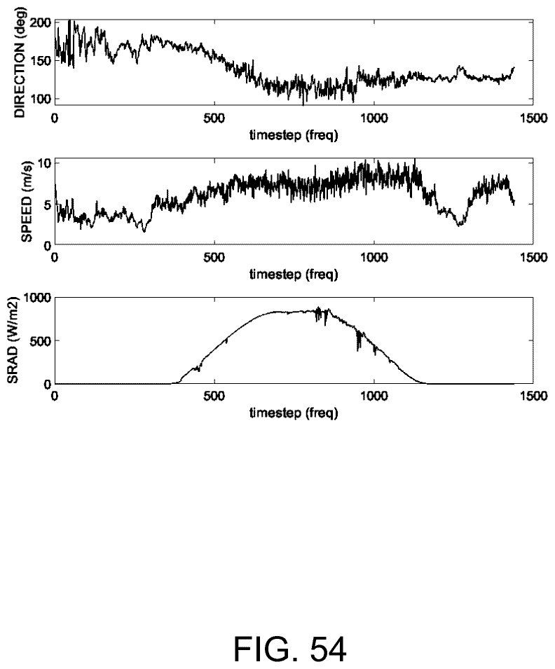

Results for a facility are illustrated in FIG. 54. In this embodiment, one particular wind realization is illustrated. In the top portion of the figure, wind direction is illustrated versus time. The middle portion of the figure illustrates wind speed and the bottom portion of the figure illustrates solar radiation. As can be seen, each of the values vary over time.

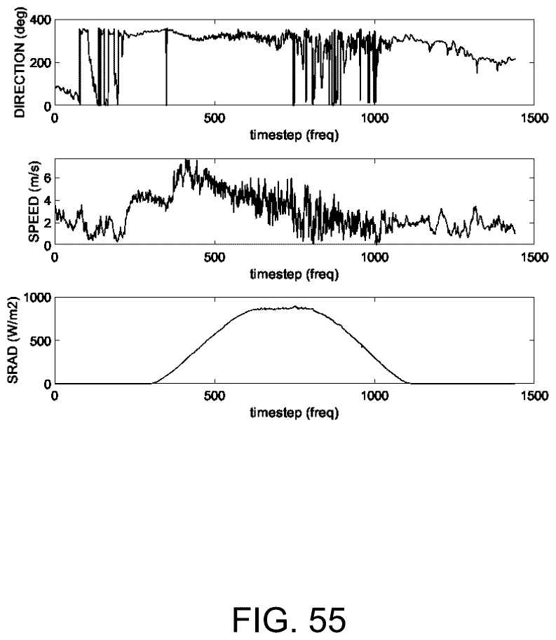

Results for a facility are illustrated in FIG. 55. In this embodiment, particular different wind realizations are illustrated. In the top portion of the figure, wind direction is illustrated versus time. The middle portion of the figure illustrates wind speed and the bottom portion of the figure illustrates solar radiation. As can be seen, each of the values vary over time and the wind realization in FIG. 55 is significantly different than that shown in FIG. 54.

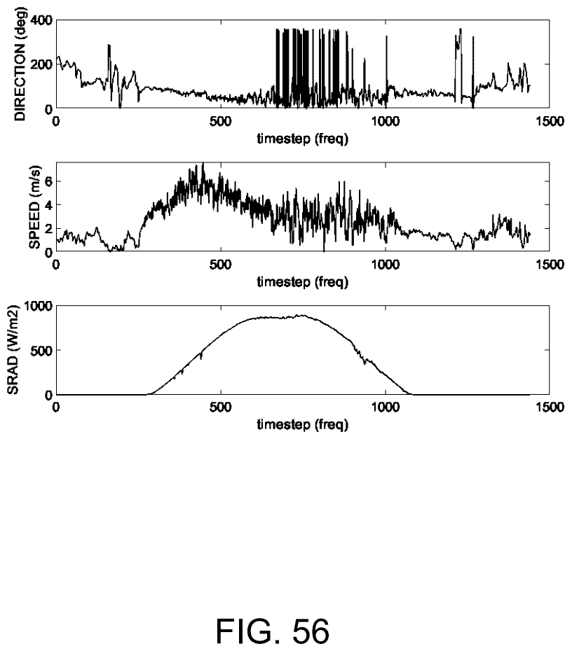

Results for a facility are illustrated in FIG. 56. In this embodiment, particular different wind realizations are illustrated. In the top portion of the figure, wind direction is illustrated versus time. The middle portion of the figure illustrates wind speed and the bottom portion of the figure illustrates solar radiation. As can be seen, each of the values vary over time and the wind realization in FIG. 56 is significantly different than that shown in FIGS. 54 and 55.

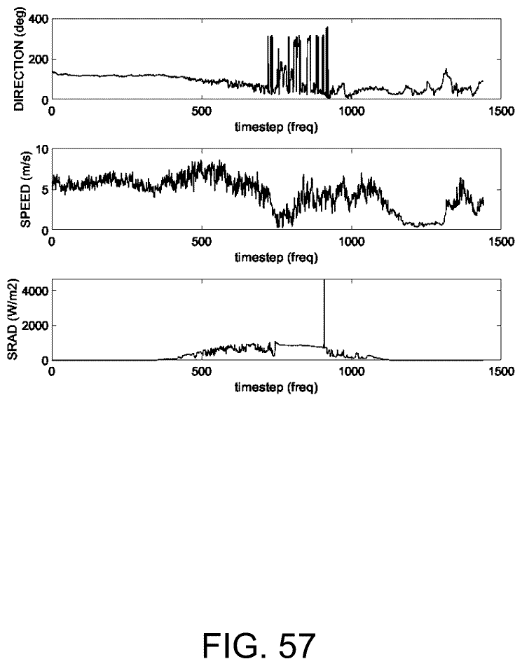

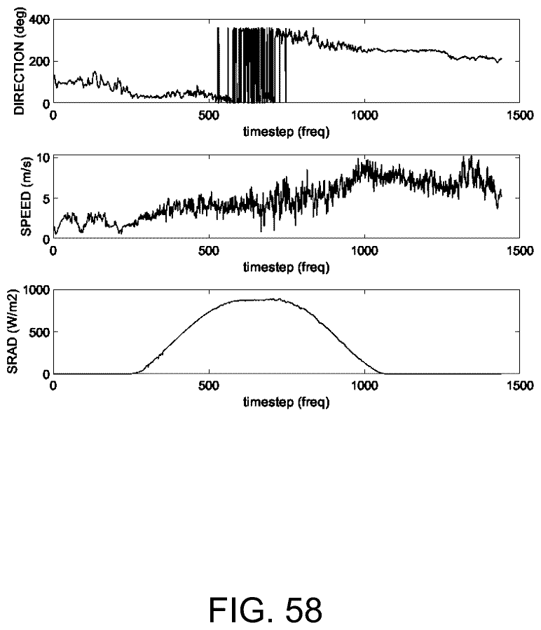

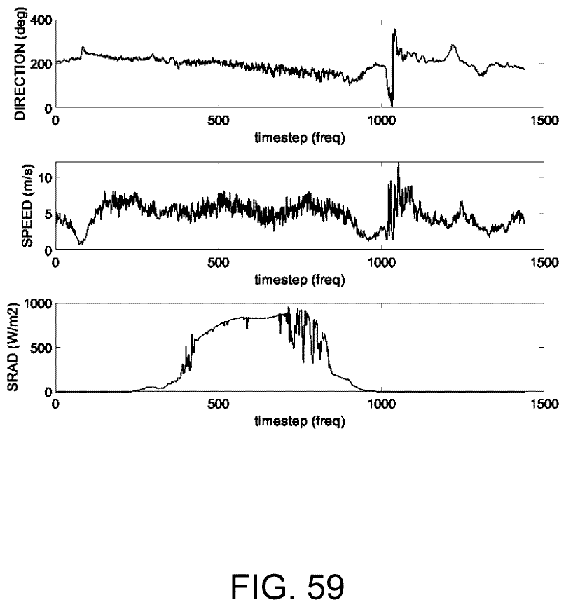

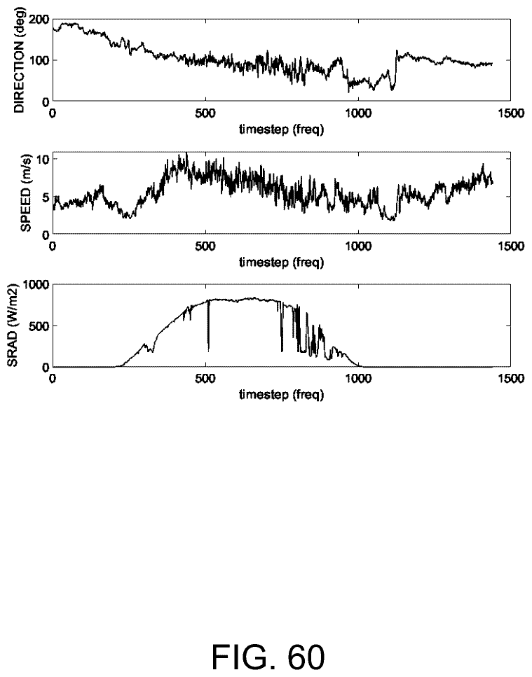

Other wind realizations are possible at the chosen site. The plotted data presents examples of prevailing wind conditions. Historical data may be used to generate stochastic wind models. In other embodiments, the wind data may be used directly, as demonstrated in FIGS. 57, 58, 59 and 60.

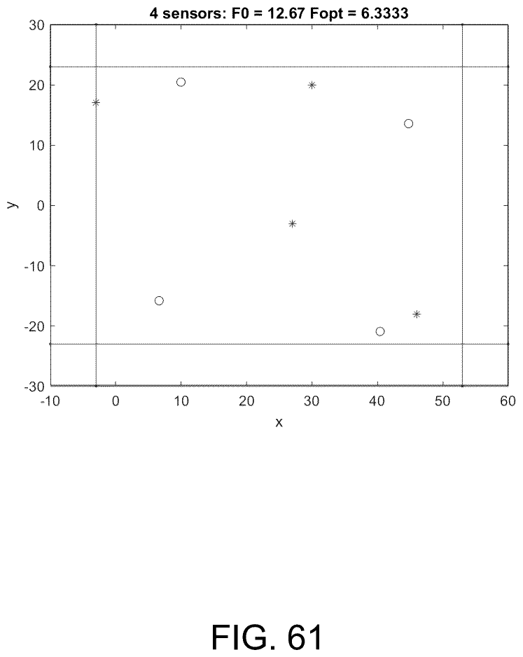

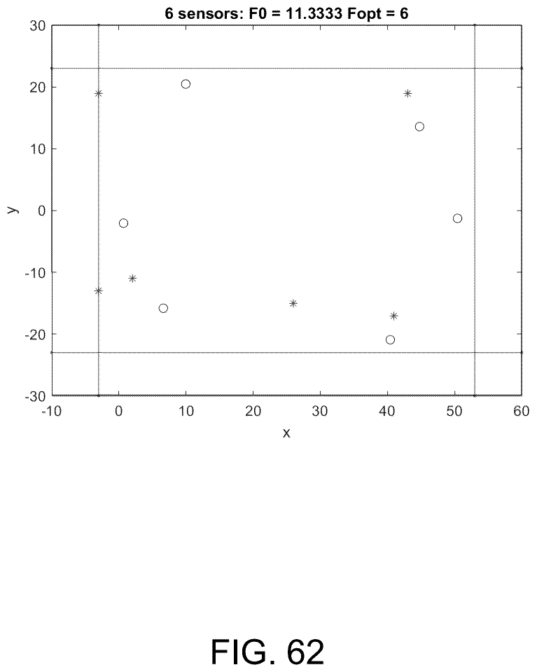

A greater number of realization may be used to provide a robust design. More stringent object measures may be used, such as the ability for sensor reading area percentages over the monitoring site. The fixed designs illustrated at the test site may be compared to the optimized designs illustrated in FIGS. 61 and 62. The fixed design with 4 sensors (blue) in FIG. 61, has a value of 12.67 percent and the optimized design (red) has a value of 6.33 percent. The fixed design with 6 sensors (blue) in FIG. 62, has a value of 11.33 percent while the optimized design (red) has a value of 6 percent. The optimized designs yield better solutions for the given set of wind realizations.

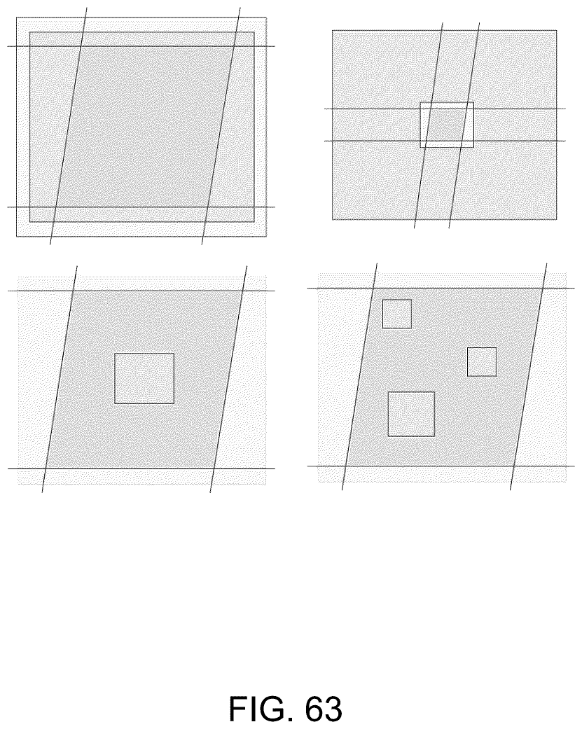

Referring to FIG. 63, a series of search and evaluation grids are illustrated. In embodiments, reduced evaluation grids allow for economy of calculations. The problem bounds stipulate the search grid as defined in yellow. The feasible search space is noted as bounded by site constraints (red lines). Evaluation regions of interest are indicated in blue. In the illustrated embodiment, the set of samples may be uniformly distributed or sampled non-uniformly. The schema illustrates that it is possible to define the search region in which sensors can be placed and that the evaluation region that is used to identify the location of potential leaks by need. This procedure provides significant flexibility for a variety of possible sites with varied restrictions. As illustrated, in the top left corner of FIG. 63, a large search area is illustrated (in yellow). On the outside of the yellow evaluation regions are located. In the top right corner, the search area is reduced while the evaluation area is larger. In the bottom left corner, the evaluation area is located within the larger search area. In the bottom right corner, multiple evaluation areas are illustrated.

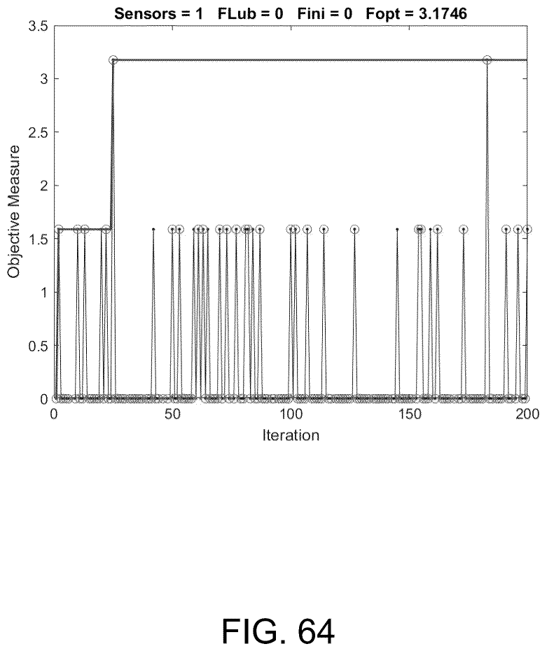

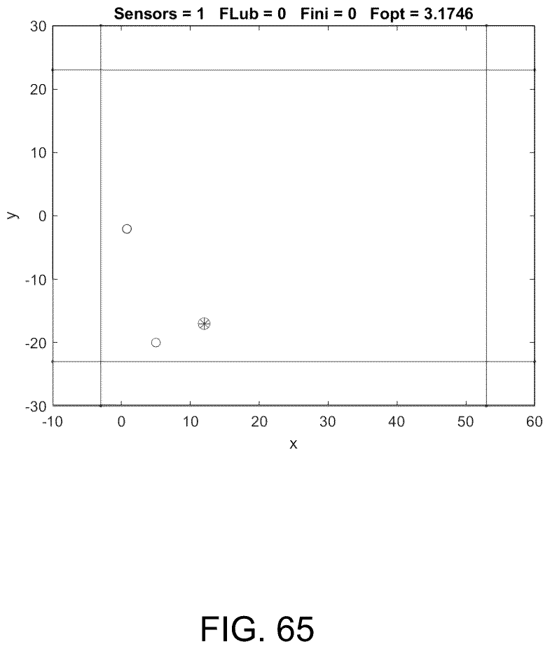

Referring to FIG. 64 an optimization profile for one sensor is illustrated with an optimized solution value of 3.17 percent. The objective measure (by iteration) is illustrated in blue with the best known solution shown in red. The fixed, initial and final solutions are illustrated in FIG. 65. FIG. 65 shows a plan view with a fixed design (shown in blue circles), an initial design (shown in red circles) and an optimal design (shown in red stars). Linear constraints are shown as red lines with the search bounds shown in black lines.

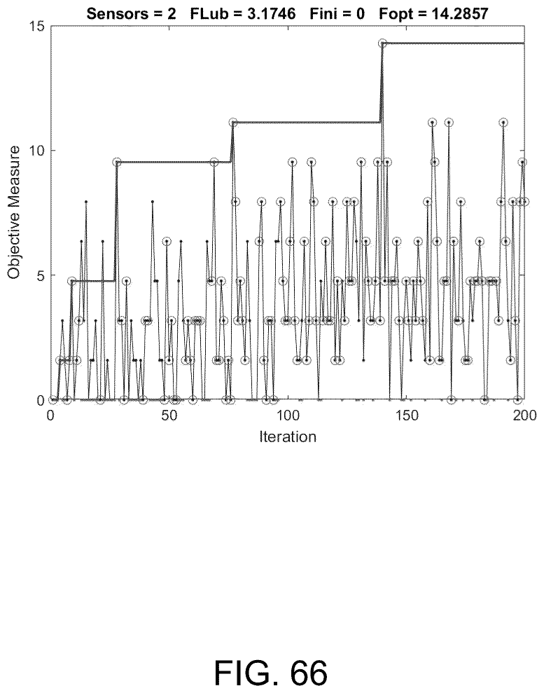

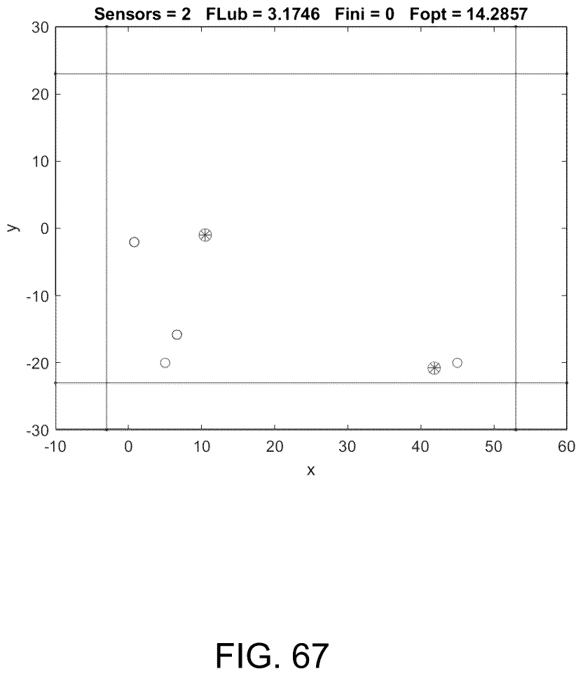

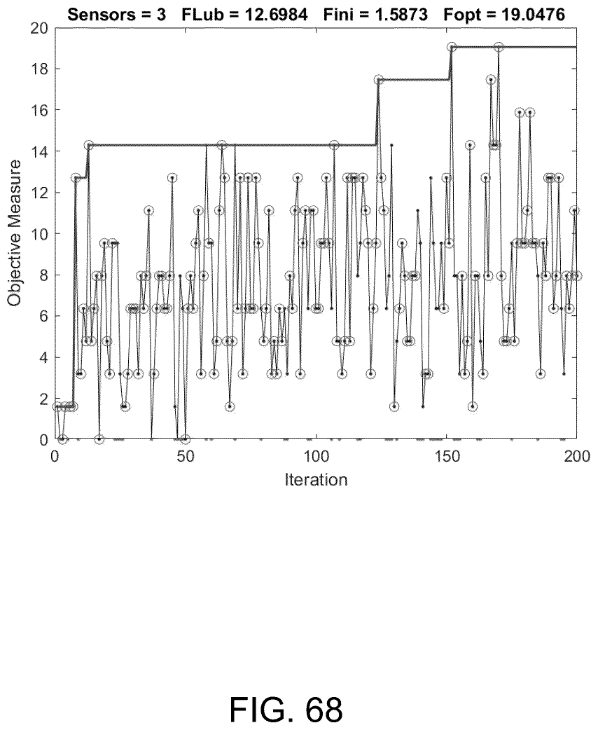

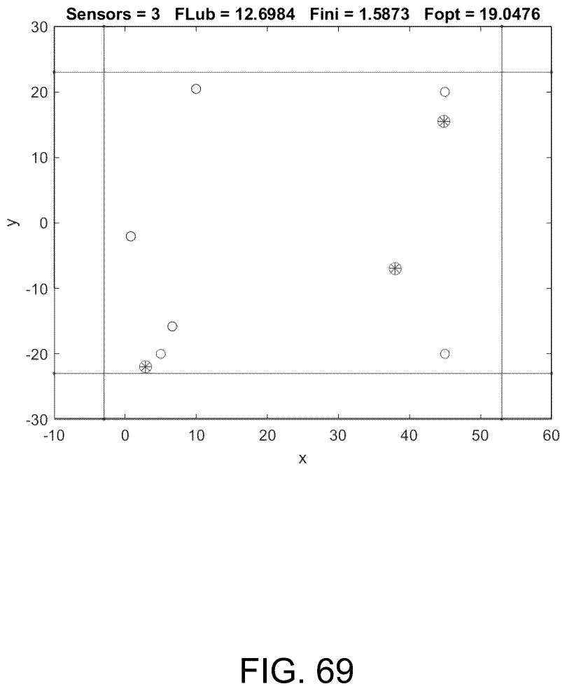

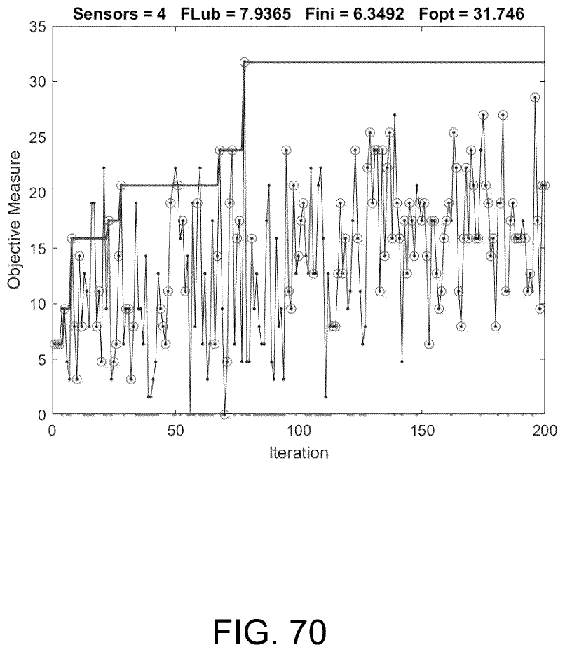

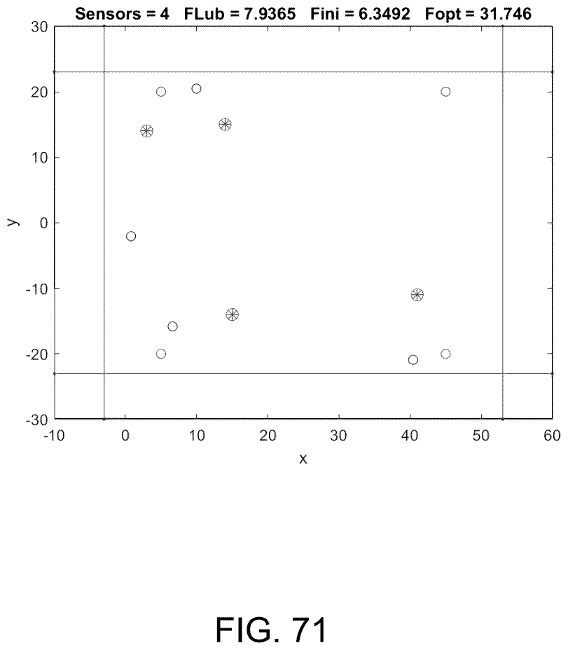

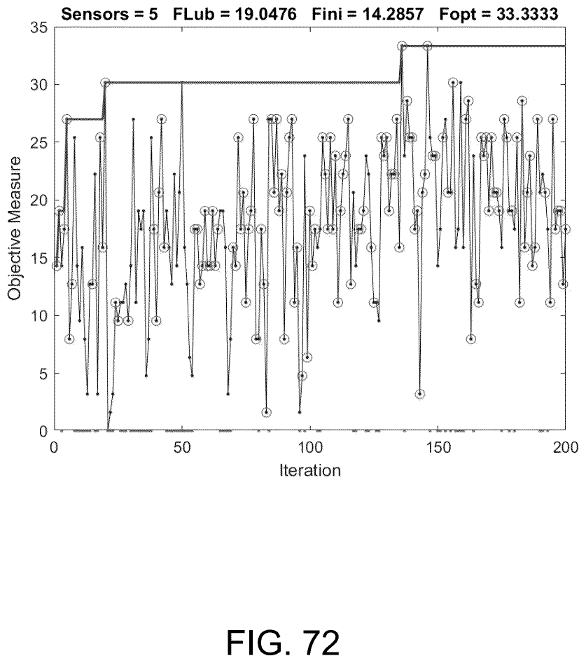

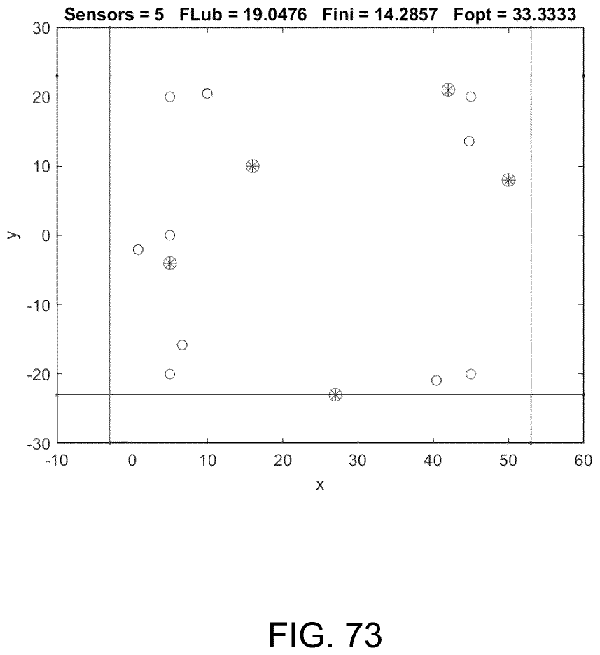

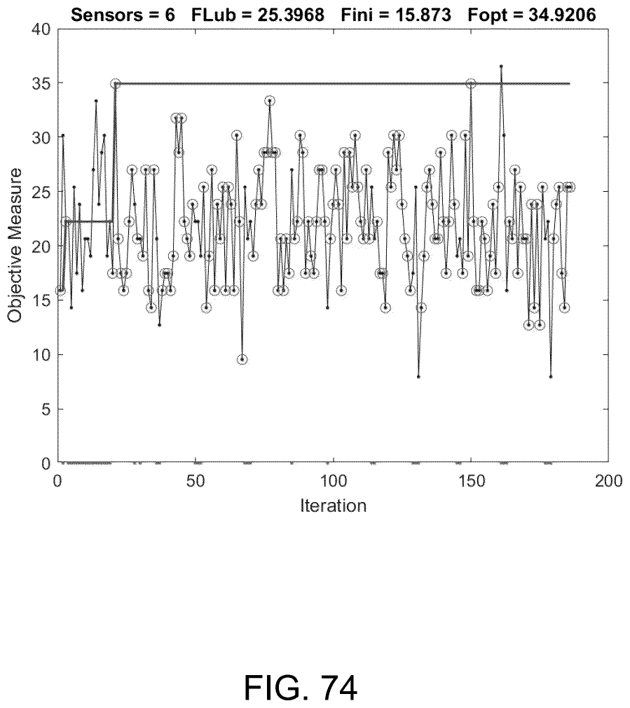

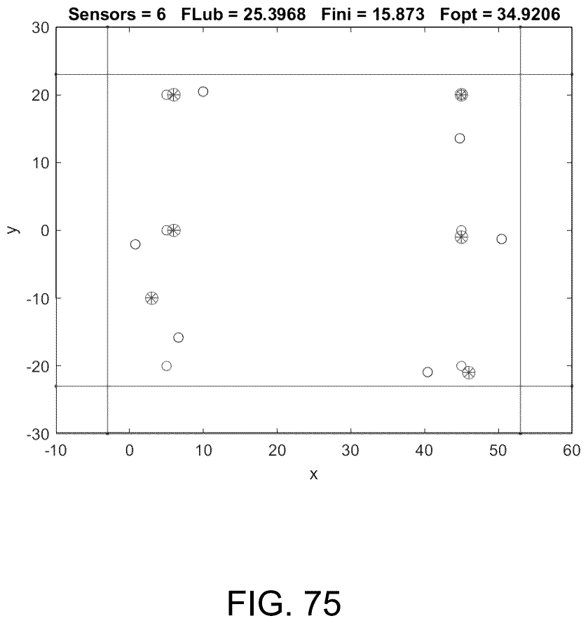

FIGS. 66 and 67 show results for two sensors. FIGS. 68 and 69 show results for three sensors. FIGS. 70 and 71 show results for four sensors. FIGS. 72 and 73 show results for five sensors. FIGS. 74 and 75 show results for six sensors.

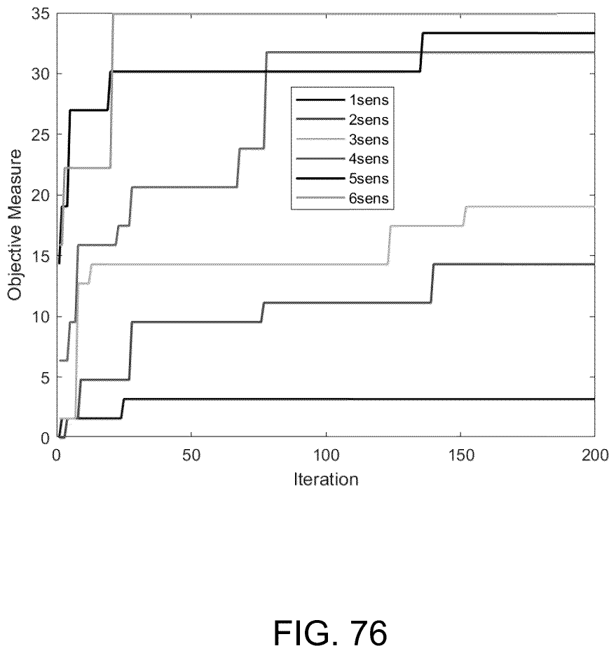

Referring to FIG. 76, the optimization profiles described above are shown together. As can be seen by the result, an increasing number of sensors improves the object measure indicative of good localization potential over the set of all seven realizations. The objective measure may saturate around a 40 percent level due to the behavior of the underlying wind profiles. In this realization, with limited wind variation, results indicate a higher optimality gap. This prevents the objective function from improving for the chosen metric.

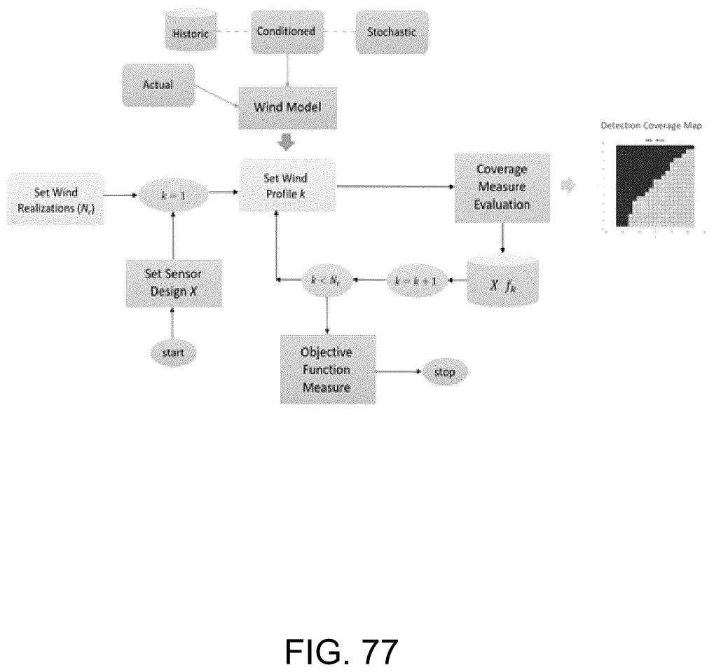

FIG. 77 shows the procedure to evaluate a sensor design X, comprising a given number of sensors (nₛ) and a set of wind realizations (nᵣ). A coverage map is established for each wind realization (as per earlier and the desired metric of interest is evaluated and stored. This may be defined as the percentage of no coverage, the percentage of good samples established (those with a low optimality gap), or percentage of samples yielding a reasonable result (solutions with low and medium gaps). The required measure of interest can be established based on the coverage map. For convenience and generality, the coverage map is specified as a uniform grid, but it may comprise a non-uniform sample set composed of potential leak source points at the given site. The intent is to provide an effective measure of the coverage (or lack thereof) for the fixed sensor design X. Once all the realizations are evaluated, the collective objective measure may be established as an aggregated quantity. One utility-based measure is to combine the mean of the responses with the standard deviation using a confidence factor as follows:

$$F ( X ) = μ ( X \vert ρ ) - γσ ( X \vert ρ )$$

where μ and σ are mean and standard deviation of the wind realization ρ with confidence factor γϵ[0 1].

Notably, each wind realization in ρ may be based on actual recorded data, or generated stochastically, conditioned to historical data of the distributions of wind speed and direction at the site. In one embodiment, wind meters may be erected at the site to record data for a minimum period of time. One realization of the wind profile may then be extracted for each day recorded and used as part of the optimal sensor placement procedure described here.

Different aspects of the disclosure are now discussed. The different aspects, as reflected in the claims, should not be considered limiting. In one example embodiment, a method is disclosed. The method may comprise obtaining wind prevailing conditions for a site and obtaining sensor data information for the site. The method may also be performed to further comprise processing the wind prevailing conditions and sensor data information to produce an event detection and record generation event and storing the event detection and record event. The method may also comprise obtaining a facility map and processing the facility map, the stored event detection and record event to produce a potential source of emissions.

In another example embodiment, the method may further comprise obtaining at least one constraint and using the at least one constraint with the solver.

In another example embodiment, the method may be performed wherein the obtaining wind prevailing conditions for the site comprises obtaining at least one of a wind speed, a wind direction, a weather for the site, a time, a temperature, a pressure, a humidity and historical information.

In another example embodiment, the method may be performed wherein the sensor data information includes a sensor type.

In another example embodiment, the method may be performed wherein the sensor data information includes a sensor GPS location.

In another example embodiment, the method may be performed wherein the sensor data information includes sensor placement information.

In another example embodiment, the method may be performed wherein the facility map comprises at least one of a site layout and a feasibility.

In another example embodiment, the method may be performed wherein the at least one constraint included at least one imposed condition.

In another example embodiment, the method may be performed wherein the processing the facility map, the stored event detection and record event to produce the potential source of emissions, includes a location.

In another example embodiment, the method may be performed wherein the processing the facility map, the stored event detection and record event to produce the potential source of emissions, includes an emission rate.

In another example embodiment, a method for identifying a source of emissions for a given site is disclosed. The method may comprise obtaining wind prevailing conditions for the site. The method may also comprise obtaining sensor data information for the site. The method may also comprise processing the wind prevailing conditions and sensor data information to produce a detection and record generation event. The method may also comprise storing the detection and record event and obtaining a facility map. The method may also comprise processing the facility map, the stored event detection, and record event to produce a potential source of emissions for the site.

In another example embodiment, the method may be performed wherein the obtaining wind prevailing conditions for the site comprises obtaining at least one of a wind speed, a wind direction, a weather for the site, a time, a temperature, a pressure, a humidity and historical information.

In another example embodiment, the method may be performed wherein the sensor data information includes a sensor type.

In another example embodiment, the method may be performed wherein the sensor data information includes a sensor GPS location.

In another example embodiment, the method may be performed wherein the sensor data information includes sensor placement information.

In another example embodiment, a method for identifying a source of methane emissions for a given site is disclosed. The method may include obtaining wind prevailing conditions for the site, wherein the wind prevailing conditions include at least one of wind direction, a wind speed, a weather for the site, a time, a temperature, a pressure and a humidity. The method may also include obtaining sensor data information for the site, wherein the sensor data information includes at least one of a sensor type, a global positioning location and data related to the sensor. The method may also comprise processing the wind prevailing conditions and sensor data information to produce a detection and record generation event. The method may also comprise storing the detection and record event on a computing apparatus. The method may also comprise obtaining a facility map, wherein the facility map includes a geographical layout and processing the facility map, the stored event detection and record event to produce a potential source of emissions for the site.

In another example embodiment, the method may be performed wherein the processing uses artificial intelligence.

In another example embodiment, a method may be performed. The method develops an emissions spatial coverage map for a facility. The method may comprise obtaining wind prevailing conditions for positions at the facility. The method may also comprise obtaining sensor data information for positions at the facility. The method may also comprise processing the wind prevailing conditions and sensor data information to produce the emissions spatial coverage map for the facility. The method may also comprise comparing the emissions spatial coverage map to a facility map. The method may also comprise determining if the emissions spatial coverage map encompasses a desired potential leak area for the facility map.

In another example embodiment, a method for developing a spatial coverage map for a site is disclosed. The method may comprise obtaining wind prevailing conditions for a site. The method may further comprise obtaining sensor data information for the site. The method may further comprise establishing an objective function measure for the site. The method may further comprise processing the wind prevailing conditions and sensor data information to produce a wind realization for the site. The method may further comprise storing the wind realization for the site. The method may further comprise establishing a coverage measure evaluation for the site based on the wind realization. The method may further comprise determining when the objective measure function has been achieved for the coverage measure evaluation. The method may further comprise ending the method when the objective function measure is successfully achieved. The method may further comprise establishing the coverage measure for a given wind realization and subsequently, establishing the mean coverage measure over all wind realizations. The method may further comprise continuing an optimization of the mean coverage measure evaluation with the given set of wind realizations until the optimal objective function measure is achieved.

In another example embodiment, the method may be performed wherein the obtaining the prevailing wind conditions is through at least one of historical weather data and sensor data being generated at the site.

In another example embodiment, the method may be performed wherein the objective function measure is a percentage of coverage of an area of the site.

In another example embodiment, the method may be performed wherein a search and evaluation grid is established over a coverage area.

In another example embodiment, the method may be performed wherein the search and evaluation grid establishes evaluation spaces and search spaces.

In another example embodiment, the method may be performed wherein the evaluation spaces and search spaces are bounded by site constraints.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may be varied in many ways. Such variations are not to be regarded as a departure from the disclosure, and all such modifications are intended to be included within the scope of the disclosure.

While embodiments have been described herein, those skilled in the art, having benefit of this disclosure, will appreciate that other embodiments are envisioned that do not depart from the inventive scope. Accordingly, the scope of the present claims or any subsequent claims shall not be unduly limited by the description of the embodiments described herein.

What is claimed is:

1. A method, comprising:
obtaining wind prevailing conditions for a site;
obtaining sensor data information for the site;
processing the wind prevailing conditions and sensor data information to produce an event detection and record generation event;
storing the event detection and record event;
obtaining a facility map; and
processing the facility map, the stored event detection, and record event to produce a potential source of emissions.

2. The method according to claim 1, further comprising:
obtaining at least one constraint; and
using the at least one constraint with the solver.

3. The method according to claim 1, wherein the obtaining wind prevailing conditions for the site comprises obtaining at least one of a wind speed, a wind direction, a weather for the site, a time, a temperature, a pressure, a humidity and historical information.

4. The method according to claim 1, wherein the sensor data information includes a sensor type.

5. The method according to claim 1, wherein the sensor data information includes a sensor global positioning satellite location.

6. The method according to claim 1, wherein the sensor data information includes sensor placement information.

7. The method according to claim 1, wherein the facility map comprises at least one of a site layout and a feasibility.

8. The method according to claim 2, wherein the at least one constraint included at least one imposed condition.

9. The method according to claim 1, wherein the processing the facility map, the stored event detection and record event to produce the potential source of emissions includes a location.

10. The method according to claim 1, wherein the processing the facility map, the stored event detection and record event to produce the potential source of emissions includes an emission rate.

11. A method for identifying a source of emissions for a given site, comprising:
obtaining wind prevailing conditions for the site;
obtaining sensor data information for the site;
processing the wind prevailing conditions and sensor data information to produce a detection and record generation event;
storing the detection and record event;
obtaining a facility map; and
processing the facility map, the stored event detection, and record event to produce a potential source of emissions for the site.

12. The method according to claim 11, wherein the obtaining wind prevailing conditions for the site comprises obtaining at least one of a wind speed, a wind direction, a weather for the site, a time, a temperature, a pressure, a humidity and historical information.

13. The method according to claim 11, wherein the sensor data information includes a sensor type.

14. The method according to claim 11, wherein the sensor data information includes a sensor global positioning satellite location.

15. The method according to claim 11, wherein the sensor data information includes sensor placement information.

16. A method for developing an emissions spatial coverage map for a facility, comprising:
obtaining wind prevailing conditions for positions at the facility;
obtaining sensor data information for positions at the facility;
processing the wind prevailing conditions and sensor data information to produce the emissions spatial coverage map for the facility;
comparing the emissions spatial coverage map to a facility map; and
determining if the emissions spatial coverage map encompasses a desired potential leak area for the facility map.

17. A method for developing a spatial coverage map for a site, comprising:
obtaining wind prevailing conditions for a site;
obtaining sensor data information for the site;
establishing an objective function measure for the site;
processing the wind prevailing conditions and sensor data information to produce a wind realization for the site;
storing the wind realization for the site;
establishing a coverage measure evaluation for the site based on the wind realization;
determining when the objective measure function has been achieved for the coverage measure evaluation;
ending the method when the objective function measure is successfully achieved;
establishing the coverage measure for a given wind realization and
subsequently, establishing the mean coverage measure over all wind realizations
then, continuing an optimization of the mean coverage measure evaluation with the given set of wind realizations until the optimal objective function measure is achieved.

18. The method for developing the spatial coverage map according to claim 17,
wherein the obtaining the prevailing wind conditions is through at least one of historical weather data and sensor data being generated at the site.

19. The method according to claim 17, wherein the objective function measure is a percentage of coverage of an area of the site.

20. The method according to claim 17, wherein a search and evaluation grid is established over a coverage area.

21. The method according to claim 20, wherein the search and evaluation grid establishes evaluation spaces and search spaces.

22. The method according to claim 21, wherein the evaluation spaces and search spaces are bounded by site constraints.
